(19) United States
(12) Patent Application Publication
(10) Pub. No.: US 2023/0149373 A1
(43) Pub. Date: May 18, 2023

(54) USE OF BROMODOMAIN INHIBITORS FOR TREATMENT OF HUNTINGTON'S DISEASE

(71) Applicant: RUMI SCIENTIFIC HOLDINGS, INC., New York, NY (US)

(72) Inventors: Carlota PEREDA SERRAS, San Francisco, CA (US); Fred ETOC, New York, NY (US)

(21) Appl. No.: 17/917,337

(22) PCT Filed: Apr. 7, 2021
(86) PCT No.: PCT/US2021/026223

§ 371 Date: Oct. 6, 2022

Related U.S. Application Data
(60) Provisional application No. 63/007,161, filed on Apr. 8, 2020.

(51) Int. Cl. A61K 31/4375; A61K 31/4709; A61K 31/4365; A61K 31/519; A61K 31/5377; A61K 31/5025

(52) U.S. Cl. CPC A61K 31/4375; A61K 31/4709; A61K 31/4365; A61K 31/519; A61K 31/5377; A61K 31/5025

(57) ABSTRACT

The present disclosure relates to the use of BRD9 inhibitors to treat Huntington's Disease.

### 1. CROSS-REFERENCE TO RELATED APPLICATIONS

This application claims the priority benefit of U.S. provisional application No. 63/007,161, filed Apr. 8, 2020, the contents of which are incorporated herein in their entireties by reference thereto.

### 2. BACKGROUND

Huntington's disease (HD) is a progressive, fatal neurodegenerative disorder that is inherited in a dominant fashion and results from a mutation that expands the polymorphic trinucleotide (CAG) tract in the Huntingtin gene (HTT). The American College of Medical Genetics/American Society of Human Genetics Huntington Disease Genetic Testing Working Group. Am J Hum Genet. 1998; 62:1243-7) indicate that 26 or fewer CAG repeats in the HTT gene are considered “normal”; 27-35 CAG repeats are considered a mutable normal allele; and 36 or greater CAG repeats are considered a disease-causing allele. The HTT gene encodes the HTT protein and the expanded CAG tract results in a pathological increase in the polyglutamine repeats near the N-terminal of the protein. It Is an autosomal dominant disease and while Individuals carry two copies of the HTT gene, one mutant allele is sufficient to result in HD.

HD is associated with a triad of motor, behavioral, and cognitive symptoms. Motor disturbances are the defining feature of the disease, with chorea the most evident motor symptom. Although useful for diagnosis, chorea is a poor marker of disease severity. Rather, disability and disease severity best correlate with negative motor features such as impairment in fine motor skills, bradykinesia, and gross motor coordination skills, including speech difficulties, gait, and postural dysfunction (Mahant et al., 2003, Neurology 61(8):1085-92).

A number of medications are prescribed to ameliorate the motor and emotional problems associated with HD; however, the scientific evidence for the usefulness of various drugs in HD is poor (Mestre et al., 2009, Cochrane Database Syst Rev. (3):CD006455; Mestre et al., 2009, Cochrane Database Syst Rev. (3):CD006456). As such, there is a significant unmet medical need to develop medications to ameliorate symptoms of HD.

### 3. SUMMARY

BRD9 is a bromodomain-containing protein that harbors a bromodomain in the amino-terminal half of its sequence, as well as a domain of unknown function (DUF3512) carboxy-terminally to it. BRD9 is part of the chromatin-remodeling BAF (also known as SWI/SNF) complex (Kadoch et al., 2013, Nat. Genet. 45, 592-601; Middeljans et al., 2012, PLoS. One. 7, e33834). Recurrent amplifications of the BRD9 locus have been observed in ovarian and breast cancer (see, e.g., Kang et al., 2008, Cancer Genet. Cytogenet. 182:1-11; Scotto et al., 2008, Mol. Cancer 7:58), and BRD9 inhibitors are being developed as potential cancer therapeutics (see, e.g., Martin et al., 2016, J. Med. Chem. 59(10):4462-4475). The present disclosure is based on the discovery that BRD9 inhibitors are effective in reversing the disease phenotype in a human organoid model of HD and thus have utility in treating patients suffering from HD.

Accordingly, the present disclosure provides methods of treating HD by administering to a subject in need thereof an effective amount of a BRD9 inhibitor. Examples of the methods and BRD9 inhibitors of use therein are described in Section 6 and specific embodiments 1 to 47, infra.

In another aspect, the present disclosure provides BRD9 inhibitors for use in the treatment of HD in a subject in need thereof. Examples of BRD9 inhibitors for use in the treatment of HD in s subject in need thereof are provided in Section 6 and specific embodiments 48 to 94, infra.

In yet another aspect, the present disclosure provides for the use of a BRD9 inhibitor in the manufacture of a medicament for the treatment of HD. Examples of using BRD9 inhibitors in the manufacture of a medicament for the treatment of HD are provided in Section 6 and specific embodiments 95 and 96, infra.

### 4. BRIEF DESCRIPTION OF THE FIGURES

FIGS. 1A-1B shows an immunofluorescence analysis of neuruloids on disk-shaped micropatterns. (FIG. 1A) Top: top view. Bottom: side view. Stained with DAPI, PAX6, and N-CAD. (FIG. 1B) left: cartoon of the ectodermal compartment within a human embryo at neurulation stages. Right: representation of a human neuruloids. Neural cells 102, neural crest 104, cranial placode 106 and epidermis 108. The reconstituted embryonic parts show a developing central nervous system organized into a neural rosette at the center (PAX6+ cells, FIG. 1A), together with neural crest (SOX10+) and placode fates (SIX1+), covered by a layer of epidermis cells (TFAP2+ only). The comparison of the in vitro neuruloid with the in vivo counterpart around day 21 post-fertilization (FIG. 1B) reveals a high level of similarities, making them an ideal pre-clinical endpoint for the study of human genetic disorders and finds drugs based on phenotypic reversal.

FIG. 2 shows the phenotypic signatures of the HD lines. (left) Representative images of PAX6 area for the different HD isogenic lines in the neuruloid assay. PAX6 staining allows visualization of the Pax6 area. (right) Associated quantification of PAX6 area normalized by the colony area. Note that the HTT −/− line showed the most dramatic phenotype. This suggests that poly-Q expansion of HTT protein represents a dominant negative loss of function, and not a gain of toxic function as it is often hypothesized.

FIG. 3 exemplifies the concept of phenotypic reversal on the HD neuruloids that will be used as the foundation of a high throughput screening campaign.

FIG. 4 shows a scheme of the AI-mediated analysis of drug screens. Specific networks are used to input all images from the screening experiment. Networks are specifically trained to output two quantities: drug toxicity and drug efficacy.

FIG. 5 shows the results of a screening campaign. The effect of 2080 compounds are plotted as a function of efficacy (phenotypic rescue) and toxicity. WT controls (RUES2) and HD-56CAG (56CAG) controls are plotted as right-pointing and left-pointing triangles, respectively. Upward-pointing triangles represent the effect of each compound. Diamonds represent hit compounds, highlighting molecules with high efficacy and low toxicity.

FIG. 6 shows that bromosporine rescues the HD neuruloids phenotype. (top) Result from the primary screen. From left to right: examples of a WT control and an HD control well as well as the HD well treated with 10 μM Bromosporine. Each well contains approximately 27 neuruloid replicates. Neuruloids are stained with: DAPI (nuclei), PAX6 (neural marker) and phalloidin (filamentous actin). (bottom) Hit validation in a small-scale experiment using Bromosporine stocks. Neuruloids are stained with: SOX10 (neural crest marker), PAX6 (neural marker) and N-CAD (cell-cell adhesion). 0.5 μM Bromosporine rescued the HD phenotype.

FIG. 7 shows quantification of Bromosporine potency and toxicity. The efficacy of Bromosporine at rescuing the neuruloids HD-56CAG phenotype is measured (open diamonds and curve). This shows an EC₅₀ of 120 nM. Bromosporine toxicity is measured as a function of concentration in the WT-20CAG background and in the HD-56CAG background.

FIG. 8 shows the activity of a panel of BRD inhibitors in the neuruloids assay at a single concentration of 10 μM. For each molecule, a dot indicates its known molecular target and both rescue efficacy (speckled bar) and toxicity levels (dashed bar) are shown. Only the compounds with rescue activity above the threshold represented by the dotted line on the right and with a toxicity below the level of the dotted line on the left are considered as hits. Only B17273, a BRD9/7 inhibitor is a hit in this experiment.

FIGS. 9A-9B shows the activity of a panel of BRD9 inhibitors in the neuruloids assay in a dose dependent manner. The potency (FIG. 9A) and toxicity (FIG. 9B) of 5 different small molecules inhibiting BRD9 are shown. All compounds are effective with a sub-micromolar EC₅₀ and show low toxicity below the micromolar range.

FIG. 10 illustrates BRD inhibitors showing sub-micromolar potency in rescuing HD neuruloids. Bromosporine, B17273, I-BRD9, dBRD9 and B19564 all rescue HD neuruloids to the WT configuration. Bromosporine is a broad spectrum inhibitor for bromodomains with IC50 of 0.41 μM, 0.29 μM, 0.122 μM and 0.017 μM for BRD2, BRD4, BRD9 and CECR2, respectively. BI-7273 is a potent, selective, and cell-permeable BRD9 BD inhibitor with IC50s of 19 nM and 117 nM for BRD9 and BRD7 respectively in alpha assay. I-BRD9 (GSK602) is a potent and selective BRD9 inhibitor with pIC50 of 7.3, while it displayed a pIC50 of 5.3 against BRD4. dBRD9 is a portent and selective BRD9 degrading PROTAC. BI-9564 is a selective inhibitor of BRD9 and BRD7 bromodomains with the IC50 of 75 nM and 3.4 μM, respectively.

FIGS. 11A-11B shows Bromosporine has HTT lowering activity. Both total HTT levels and expanded HTT levels were measured in neuruloids treated with DMSO control (conc 0), 5 μM (conc 1) or 1 μM (conc 2) Bromosporine, B17273, dBRD9 or BI9564. The assay was performed in two genetic background: 56CAG (FIG. 11A) and 72CAG (FIG. 11B). The dotted line on each graph refers to control levels of HTT in the DMSO treated control. The value of the signal measured by the MSD assay is specific to the antibody used and is therefore different in the case where expanded HTT was measured of total HTT since these two measurements are made with different antibodies, hence the absolute levels in the two measures cannot be compared.

FIGS. 12A-12B shows BRD inhibitors rescue HD gastruloids. (FIG. 12A) Gastruloids are created by application of CHIR and Activin for two days on pluripotent micropatterned cultures. In the WT-20CAG configuration, a SOX17+ ring is forming at the colony periphery. This ring is enlarged in the HD-56CAG background and dramatically takes over the full colony in the knock out HTT−/− background. (FIG. 12B) Quantification of the SOX17+ rings in gastruloids treated with BRD inhibitors. All treatments show a reduction in the area of the SOX17+ ring compared to the WT-20CAG background.

FIGS. 13A-13C shows BRD9 knockdown partially rescuing HD-56CAG neuruloids. (FIG. 13A) Inducible CRISPR interference construct lowers BRD9 mRNA levels by 50%. (FIG. 13B) Relative to WT-20CAG neuruloids (right), HD-56CAG (left) shows an expanded PAX6 area and a smaller number of SOX10+ cells. Both of these features are partially rescued by BRD9 knockdown (middle). (FIG. 13C) Associated quantifications. N>40 colonies for each condition.

### 5. DEFINITIONS

Administer: The terms “administer,” “administering,” or “administration” refers to introducing a compound or pharmaceutical composition to a subject, for example, by subcutaneous injection, intraperitoneal injection, intramuscular injection, intravenous injection, epidermal or transdermal administration, mucosal membrane administration, orally, nasally, rectally, or vaginally. Targeting of the compounds and pharmaceutical compositions to the tissues of the central nervous system may involve delivery to the CSF and brain by intrathecal, intracerebroventricular or intraparenchymal administration. Carrier formulations may be selected or modified according to the route of administration. As a general reference, see, for example, Remington—The Science and Practice of Pharmacy, 21^{st} edition. Gennaro et al. editors. Lippincott Williams & Wilkins Philadelphia.

BRD9 Inhibitor: The term “BRD9 inhibitor” refers to a compound that inhibits the activity of BRD9. In some embodiments, the BRD9 inhibitor is a broad spectrum bromodomain inhibitor with activity against one or more bromodomain proteins in addition to BRD9. In some embodiments, the BRD9 inhibitor is a selective inhibitor of BRD9. For example, a BRD9 inhibitor can have at least two-fold, at least five-fold or at least ten-fold greater activity against BRD9 vs. one, two, or three other bromodomain-containing proteins, such as, but not limited to, BRD2, BRD3, BRD4, or any combination of the foregoing. Because BRD9 and BRD7 are closely related, in certain embodiments, a selective inhibitor of BRD9 can inhibit BRD7 to a similar degree as BRD9, provided that it has lesser inhibitory activity on BRD2, BRD3 and/or BRD4.

Bromodomain: The term “bromodomain” refers to a protein domain that recognizes acetylated lysine residues such as those on the N-terminal tails of histones. In certain embodiments, a bromodomain, e.g., of a bromodomain-containing protein (e.g., bromo and extra terminal (BET) protein), comprises about 110 amino acids and shares a conserved fold comprising a left-handed bundle of four alpha helices linked by diverse loop regions that interact with chromatin. In certain embodiments, the bromodomain is ASH1L (GenBank ID: gi|8922081), ATAD2 (GenBank ID: gi|24497618), BAZ2B (GenBank ID: gi|7304923), BRD1 (GenBank ID: gi|11321642), BRD2(1) (GenBank ID: gi|4826806), BRD2(2) (GenBank ID: gi|4826806), BRD3(1) (GenBank ID: gi|11067749), BRD3(2) (GenBank ID: gi|11067749), BRD4(1) (GenBank ID: gi|19718731), BRD4(2) (GenBank ID: gi|19718731), BRD9 (GenBank ID: gi|57770383), BRDT(1) (GenBank ID: gi|46399198), BRPF1 (GenBank ID: gi|51173720), CECR2 (GenBank ID: gi|148612882), CREBBP (GenBank ID: gi|4758056), EP300 (GenBank ID: gi|50345997), FALZ (GenBank ID: gi|38788274), GCN5L2 (GenBank ID: gi|10835101), KIAA1240 (GenBank ID: gi|51460532), LOC93349 (GenBank ID: gi|134133279), PB1(1) (GenBank ID: gi|30794372), PB1(2) (GenBank ID: gi|30794372), PB1(3) (GenBank ID: gi|30794372), PB1(5) (GenBank ID: gi|30794372), PB1(6) (GenBank ID: gi|30794372), PCAF (GenBank ID: gi|140805843), PHIP(2) (GenBank ID: gi|34996489), SMARCA2 (GenBank ID: gi|48255900), SMARCA4 (GenBank ID: gi|21071056), SP140 (GenBank ID: gi|52487219), TAF1(1) (GenBank ID: gi|20357585), TAF1(2) (GenBank ID: gi|20357585), TAF1L(1) (GenBank ID: gi|24429572), TAF1L(2) (GenBank ID: gi|24429572), TIF1 (GenBank ID: gi 14971415), TRIM28 (GenBank ID: gi|5032179), or WDR9(2) (GenBank ID: gi|16445436).

Degron: The term “degron” refers to a sequence of amino acids that provides a degradation signal that directs a polypeptide for cellular degradation. The degron may promote degradation of an attached polypeptide through either the proteasome or autophagy-lysosome pathways. See, e.g., Kanemaki et al., 2013, Pflugers Arch. 465(3):419-425 and Erales et al., 2014, Biochim Biophys Acta 1843(1):216-221.

Dendrimer: The term “dendrimer” as used herein is intended to include, but is not limited to, a molecular architecture with an interior core, interior layers (or “generations”) of repeating units regularly attached to this initiator core, and an exterior surface of terminal groups attached to the outermost generation. Examples of dendrimers include, but are not limited to, poly(amidoamine) (PAMAM), polyester, polylysine, and poly(propylene imine) (PPI). The PAMAM dendrimers can have carboxylic, amine and hydroxyl terminations and can be any generation of dendrimers including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. Dendrimers suitable for use with the present invention include, but are not limited to, polyamidoamine (PAMAM), polypropylamine (POPAM), polyethylenimine, polylysine, polyester, iptycene, aliphatic poly(ether), and/or aromatic polyether dendrimers. Each dendrimer of the dendrimer complex may be of similar or different chemical nature than the other dendrimers (e.g., the first dendrimer may include a PAMAM dendrimer, while the second dendrimer may comprises a POPAM dendrimer). In some embodiments, the first or second dendrimer may further include an additional agent. The multiarm PEG polymer includes a polyethylene glycol having at least two branches bearing sulfhydryl or thiopyridine terminal groups; however, embodiments disclosed herein are not limited to this class and PEG polymers bearing other terminal groups such as succinimidyl or maleimide terminations can be used. The PEG polymers in the molecular weight 10 kDa to 80 kDa can be used.

Inhibition: The term “inhibition,” “inhibiting,” “inhibit,” or “inhibitor” refer to the ability of a compound to reduce, slow, halt or prevent activity of a particular protein or biological process (e.g., activity of a bromodomain and/or a bromodomain-containing protein). In some embodiments the activity is reduced in a cell or tissue and/or the activity is reduced relative to vehicle.

Neuruloid: The term “neuruloid” refers to a self-organized organoid on a micropattern harboring neural progenitors, neural crest, sensory placode and epidermis. Neuruloids can be generated from embryonic stem cells, e.g., human embryonic stem cells, as described by Harekami et al., 2019, Nature Biotechnology 37:1198-1208.

Selective Inhibition: As used herein, when a compound has the ability to “selectively” or “specifically” reduce the activity of BRD9 as compared to one or more other bromodomain-containing proteins, the compound can inhibit the activity of BRD9 as compared to another bromodomain by at least about 2-fold. In various embodiments, the compound has a greater inhibitory activity on BRD9 as compared to another bromodomain other than BRD7 by at least about 5-fold, at least about 10-fold, at least about 25-fold, or at least about 50-fold. The inhibitory activity can be measured as a % inhibition of activity and/or an IC₅₀ value in in vitro assays as described in Section 6. Because BRD9 and BRD7 are closely related, in certain embodiments, a selective of inhibitor of BRD9 can inhibit BRD7 to a similar degree as BRD9, provided that it has lesser inhibitory activity on one or more distantly related bromodomain-containing proteins, such as BRD2, BRD3 and/or BRD4.

Subject or Patient: The terms “subject” and “patient” and refer to a human (i.e., male or female of any age group, e.g., pediatric subject (e.g., infant, child, or adolescent) or adult subject (e.g., young adult, middle-aged adult, or senior adult)) or non-human animal (e.g., a mammal such as a non-human primate, a domestic animal such as a cat or dog, or livestock animal such as a cow, horse or pig). In particular embodiments, the subject has an expanded polyglutamine or polyQ repeat in at least one HTT allele. The expanded polyglutamine repeats may comprise one or both codons for glutamine (i.e., CAA and/or CAG) and encode an HTT protein with 36 or more, and preferably 40 or more glutamines. In particular embodiments, the polyglutamine repeat encodes an HTT protein with 42 to 265 glutamines, and in certain specific embodiments 45, 48, 50, 55, 56, 58, 60, 65, 67, 70, 72, 74, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250 or 265 glutamines, and any derivable range therein having as endpoints two of the foregoing glutamine repeat numbers, e.g., 48 to 180, 50 to 150, or 56 to 130 glutamines. Because HD is an autosomal dominant condition, the subject may have an expanded glutamine repeat in only one HTT allele, but subjects with expanded glutamine repeats in both HTT alleles are within the scope of this disclosure.

Treat: The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a family history of HD or the presence of expanded glutamine or CAG repeats in the Huntingtin gene).

### 6. DETAILED DESCRIPTION

BRD9 inhibitors are known in the art and can be used in the treatment of Huntington's Disease (HD). BRD9 inhibitors may be of varied nature and origin, including but not limited to nucleic acids, polypeptides or small molecules.

In one aspect the inhibitor is an antisense nucleic acid capable of inhibiting transcription of the BRD9 gene or translation of the BRD9 mRNA. The antisense nucleic acid can comprise all or part of the sequence encoding the bromodomain-containing protein, or of a sequence that is complementary thereto. The antisense sequence can be a DNA, an RNA (e.g., siRNA), a ribozyme, etc. It may be single-stranded or double stranded. It can also be an RNA encoded by an antisense gene. When an antisense nucleic acid comprising part of the sequence of the gene or mRNA is being used, it is preferred to use a part comprising at least 10 consecutive bases from the sequence, more preferably at least 15 consecutive bases from the sequence, in order to ensure specific hybridization. In the case of an antisense oligonucleotide, it typically comprises fewer than 100 bases, for example in the order of 10 to 50 bases or 18 to 30 bases. An antisense oligonucleotide can be modified to improve its stability, its nuclease resistance, its cell penetration, etc. Perfect complementarily between the sequence of the antisense molecule and that of the BRD9 gene or mRNA is not required, but is generally desired.

In other embodiments, the BRD9 inhibitor is a polypeptide or peptide. It may be, for example, a peptide comprising a region of the bromodomain-containing protein, and capable of antagonizing the bromodomain-containing protein's activity. A peptide advantageously comprises from 5 to 50 consecutive amino acids of the primary sequence of the BRD9 protein, typically from 7 to 40. The polypeptide can also be an antibody against the bromodomain-containing protein, or a fragment or derivative of such an antibody, for example a Fab fragment or a single chain antibody (e.g., ScFv). Such antibodies, fragments, or derivatives can be produced by conventional techniques.

In yet other embodiments, the BRD9 inhibitor is a small molecule. BRD9 inhibitors include but are not limited to I-BRD9, TP-472, BI-7273, BI-9564, dBRD9, GNE-375 and LP-99, methylquinolinone compounds, thienopyridone as well as BRD9 inhibitors disclosed in Remillard et al., 2017, Angew. Chem. Int. Ed. 56:1-7 and Theodoulou et al., 2016, J. Med. Chem. 99:1425-39 (all herein incorporated by reference). Small molecule BRD9 inhibitors can be administered in the form of free bases or physiologically acceptable salts.

In a specific embodiment, the BRD9 inhibitor is BI-9564:

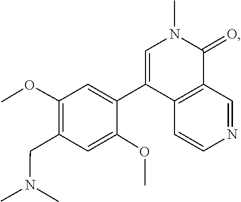 [SMILES: COc1cc(-c2cn(C)c(=O)c3cnccc23)c(OC)cc1CN(C)C]

or a pharmaceutically acceptable salt thereof.

In another specific embodiment, the BRD9 inhibitor is BI-7273:

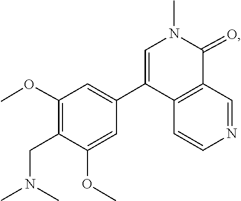 [SMILES: COc1cc(-c2cn(C)c(=O)c3cnccc23)cc(OC)c1CN(C)C]

or a pharmaceutically acceptable salt thereof.

In another specific embodiment, the BRD9 inhibitor is LP-99:

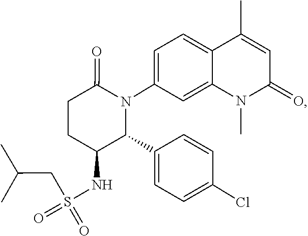 [SMILES: Cc1cc(=O)n(C)c2cc(N3C(=O)CC[C@H](NS(=O)(=O)CC(C)C)[C@H]3c3ccc(Cl)cc3)ccc12]

or a pharmaceutically acceptable salt thereof.

In another specific embodiment, the BRD9 inhibitor is I-BRD9:

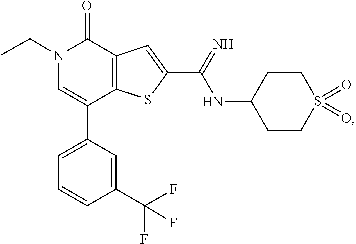 [SMILES: CCn1cc(-c2cccc(C(F)(F)F)c2)c2sc(C(=N)NC3CCS(=O)(=O)CC3)cc2c1=O]

or a pharmaceutically acceptable salt thereof.

In yet another specific embodiment, the BRD9 inhibitor is d-BRD9:

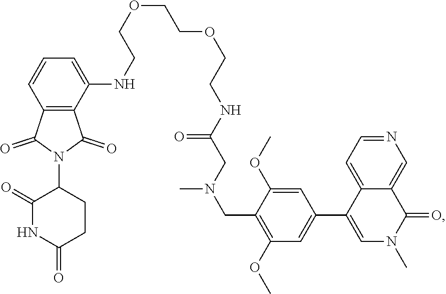 [SMILES: COc1cc(-c2cn(C)c(=O)c3cnccc23)cc(OC)c1CN(C)CC(=O)NCCOCCOCCNc1cccc2c1C(=O)N(C1CCC(=O)NC1=O)C2=O]

or a pharmaceutically acceptable salt thereof.

In yet a further specific embodiment, the BRD9 inhibitor is TP-472:

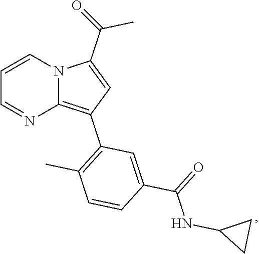 [SMILES: CC(=O)c1cc(-c2cc(C(=O)NC3CC3)ccc2C)c2ncccn12]

or a pharmaceutically acceptable salt thereof.

In yet a further specific embodiment, the BRD9 inhibitor is GNE-375:

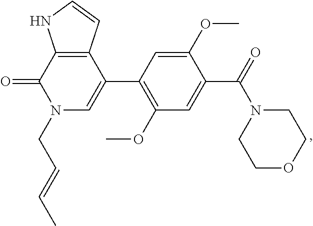 [SMILES: C/C=C/Cn1cc(-c2cc(OC)c(C(=O)N3CCOCC3)cc2OC)c2cc[nH]c2c1=O]

or a pharmaceutically acceptable salt thereof.

In yet further specific embodiments, the BRD9 inhibitor is a compound of Formula (I):

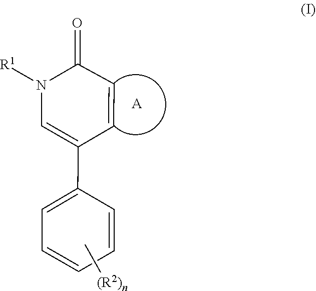

or an enantiomer, diastereomer, stereoisomer, or pharmaceutically acceptable salt thereof, wherein:
A is phenyl or 5- or 6-membered heteroaryl containing 1 or 2 heteroatoms selected from N and S, wherein the phenyl or heteroaryl is unsubstituted or substituted with 1 to 3 R³ groups;
R¹ is H, (C₁-C₄)alkyl, or (C₁-C₄)haloalkyl;
each R² is independently (C₁-C₄)alkyl, (C₁-C₄)haloalkyl, (C₁-C₄)alkoxy, (C₁-C₄)haloalkoxy, halogen, OH, or NH₂;
each R³ is independently (C₁-C₄)alkyl, (C₁-C₄)haloalkyl, (C₁-C₄)alkoxy, (C₁-C₄)haloalkoxy, halogen, OH, NH₂, or

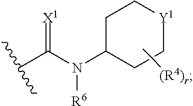

X₁ is NR⁵ or O;
Y₁ is S(O)ₐ or NR⁵;
each R⁴ is independently (C₁-C₄)alkyl, (C₁-C₄)haloalkyl, halogen, or —C(O)(C₁-C₃)alkyl;
each R⁵ is independently H or (C₁-C₄)alkyl;
each R⁶ is independently H or (C₁-C₄)alkyl;
a is 0, 1, or 2; and
n and r are each independently 0, 1, 2, or 3.

In yet further specific embodiments, the BRD9 inhibitor is a compound of Formula (II):

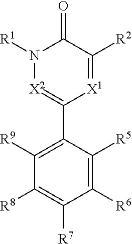

### (II)

or an enantiomer, diastereomer, stereoisomer, or pharmaceutically acceptable salt thereof, wherein:
R¹ is (C₁-C₃)alkyl or cyclopropyl;
R² is halogen, (C₁-C₃)alkyl, (C₁-C₃)haloalkyl, NH₂, NH(C₁-C₃)alkyl or OH;
X¹ is N or CR³, and X² is N or CR⁴; provided that X¹ and X² cannot be both N;
R³ is H or (C₁-C₃)alkyl;
R⁴ is H or (C₁-C₃)alkyl; provided that R³ and R⁴ cannot be both (C₁-C₃)alkyl; alternatively, R² and R³ taken together form a benzene ring or a 5-6 membered heteroarene ring, each of which rings can be independently unsubstituted or substituted with one or more groups which are independently halogen, OH, NH₂, NH(C₁-C₃)alkyl or (C₁-C₃)alkyl, wherein the (C₁-C₃)alkyl group can be unsubstituted or substituted with 5-6 membered heteroaryl or phenyl;
R⁵ and R⁹ are the same or different and are independently H, O(C₁-C₃)alkyl or (C₁-C₃)alkyl;
R⁶ and R⁸ are the same or different and are independently H, OH, halogen, NH₂, (C₁-C₃)alkyl, O(C₁-C₃)alkyl, O(C₁-C₃) haloalkyl, (C₁-C₃)alkyl-O—(C₁-C₃)alkyl, 4-7 membered heterocycloalkyl, (C₁-C₃)alkyl-SO₂—(C₁-C₃)alkyl, (C₁-C₃)alkyl-NH₂, (C₁-C₃)alkyl-N((C₁-C₃)alkyl)₂, N((C₁-C₃)alkyl)₂, or NHR¹³;
R¹³ is independently for each occurrence SO₂—(C₁-C₃)alkyl or (C₁-C₃)alkyl, wherein the (C₁-C₃)alkyl groups are unsubstituted or substituted with 5 to 6 membered heteroaryl;
alternatively, R⁵ and R⁶ taken together form a benzene ring;
alternatively, R⁷ and R⁶ or R⁷ and R⁸ taken together form a 5-7 membered heterocycloalkyl which is unsubstituted or substituted with (C₁-C₃)alkyl;
R⁷ is H, NH₂, Y—R¹², (C₁-C₃)alkyl or 4-7 membered heterocycloalkyl;
Y is CR¹⁰R¹¹, SO₂ or CO;
R¹⁰ and R¹¹ are the same or different and are independently H or (C₁-C₃)alkyl; or
R¹⁰ and R¹¹ taken together form a C₃₋₄ cycloalkyl;
R¹² is NH₂, OH, (C₁-C₃)alkyl, N(R¹⁵,R¹⁶), OR¹⁷, aryl, or 5-6 membered heteroaryl, wherein the aryl or heteroaryl are independently unsubstituted or substituted with one or more halogen or 4-7 membered heterocycloalkyl, each of which heterocycloalkyl is independently unsubstituted or substituted with one or more groups selected from halogen, OH, NH₂, (C₁-C₃)alkyl, NH(C₁-C₃)alkyl, N((C₁-C₃)alkyl)₂, O(C₁-C₃)alkyl and CH₂R¹⁴;
R¹⁴ is 5-10 membered mono- or bicyclic aryl or heteroaryl, which is unsubstituted or substituted with NH₂, OH, halogen, CN, (C₁-C₃)alkyl, or O(C₁-C₃)alkyl;
R¹⁵ is H or (C₁-C₃)alkyl;
R¹⁶ is (C₁-C₃)alkyl, C₂₋₃ alkyl-N((C₁-C₃)alkyl)₂, C₂₋₃alkyl-NH(C₁-C₃)alkyl or 4-7 membered heterocycloalkyl, which heterocycloalkyl is unsubstituted or substituted with (C₁-C₃)alkyl;
R¹⁷ is (C₁-C₃)alkyl or 4-7 membered heterocycloalkyl, which heterocycloalkyl is unsubstituted or substituted with (C₁-C₃)alkyl;
wherein when R⁷ is YR¹², R⁶ and R⁸ can be the same or different and are independently H, OH, halogen, NH₂, CN, (C₁-C₃)alkyl, (C₁-C₃) haloalkyl, O(C₁-C₃)alkyl, O(C₁-C₃) haloalkyl or (C₁-C₃)alkyl-O—(C₁-C₃)alkyl; and
wherein at least one of the substituents R⁵ to R⁹ is not hydrogen.

In yet a further specific embodiment, the BRD9 inhibitor is bromosporine:

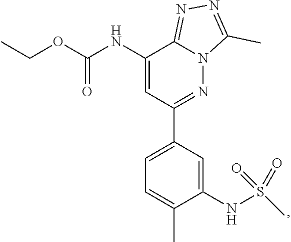 [SMILES: CCOC(=O)Nc1cc(-c2ccc(C)c(NS(C)(=O)=O)c2)nn2c(C)nnc12]

or a pharmaceutically acceptable salt thereof.

The disclosure is not limited to particular BRD9 antagonists. Suitable BRD9 inhibitors can decrease BRD9 activity by at least about 10%, at least about 20%, at least about 30%, at least about 40%, at least about 50%, at least about 60%, at least about 70%, at least about 80%, at least about 90%, at least about 95%, or at least about 98%. In certain embodiments, the activity of a BRD9 inhibitor decreases BRD9 activity by at most about 90%, at most about 80%, at most about 70%, at most about 60%, at most about 50%. Ranges combining any pair of the foregoing values (e.g., from at least about 30% to at most about 90% or from at least about 50% to at most 90%) are also within the scope of the disclosure.

Methods of determining BRD9 inhibitory activity are known. For example, the inhibitory activity can be determined using the TR-FRET methods described in Theodoulou et al., 2016, J. Med. Chem. 99: 1425-39. Briefly, compounds can be incubated with Alexa Fluor647 ligand (GSK2833930A) in Greiner 384-well black low volume microtiter plates and incubated in the dark for 30 min at room temperature. Detection reagents can include Eu-W1024 Anti-6xHis antibody. The plates can be read to determine donor and acceptor counts. From this, the ratio of acceptor/donor is calculated (Δex=337 nm, Δem donor=615 nm, em acceptor=665 nm) and used for data analysis. In various embodiments, antagonists can decrease BRD9 activity by at least 20%, at least 50%, at least 75%, at least 80%, at least 90%, at least 100%, at least 200%, or even at least 1000% or more as compared to the absence of the inhibitor. The assay can be adapted to assay the inhibitory effect of a given BRD9 inhibitor on other bromodomain-containing proteins to assess the selectivity for BRD9. A selective BRD9 inhibitor can have at least two-fold, at least five-fold or at least ten-fold greater % inhibition against BRD9 vs. one, two, or three other bromodomain-containing proteins, such as, but not limited to, BRD2, BRD3, BRD4, or any combination of the foregoing. In the case of BRD4, which has two binding domains (Binding Domain 1, or BD1, and Binding Domain 2, BD2), a single residue mutation in the BD2 acetyl lysine binding pocket (Y390A) can be introduced to lower the affinity of the fluoroligand for the mutated BD2 domain in order to determine the binding of BRD9 inhibitors to the single non-mutated BD1 bromodomain.

Alternatively the inhibitory activity of BRD9 inhibitors can be evaluated as follows. His/Flag epitope tagged BRD9₁₃₄₋₂₃₉ is cloned, expressed, and purified to homogeneity. BRD9 binding and inhibition can be assessed by monitoring the engagement of biotinylated H4-tetraacetyl peptide (New England Peptide, NEP2069-11/13) with the target using the AlphaLisa technology (Perkin-Elmer). Specifically, in a 384 well ProxiPlate BRD9 (50 nM final) is combined with peptide (3 nM final) in 50 mM HEPES (pH 7.5), 150 mM NaCl, 1 mM TCEP, 0.01% (w/v) BSA, and 0.008% (w/v) Brij-35 either in the presence of DMSO (final 0.8% DMSO) or compound dilution series in DMSO. After 20 minutes incubation at room temperature AlphaLisa Streptavidin Acceptor Beads (Perkin-AL125C) and AlphaLisa Nickel donor beads (Perkin AS 10 ID) are added to a final concentration of 15 μg/mL each. After ninety minutes of equilibration in the dark, the plates are read on an Envision instrument and IC₅₀s calculated using a four-parameter non-linear curve fit. The assay can be adapted to assay the inhibitory effect of a given BRD9 inhibitor on other bromodomain-containing proteins to assess the selectivity for BRD9. A selective BRD9 inhibitor can have at least two-fold, at least five-fold or at least ten-fold lower IC₅₀ against BRD9 vs. one, two, or three other bromodomain-containing proteins, such as, but not limited to, BRD2, BRD3, BRD4, or any combination of the foregoing.

A BRD9 inhibitor can be tested for its ability to reverse the HD neuruloid phenotype. Neuruloids are micropattern-based self-organized cellular assemblies of ectodermal origin that mimic neurulation (Harekami et al., 2019, Nature Biotechnology 37:1198-1208). These neuruloids show in particular a developing central nervous system organized into a neural rosette at the center. The comparison of the in vitro neuruloid with the in vivo counterpart around day 21 post-fertilization reveals a high level of similarity, making them an ideal pre-clinical endpoint for the study of human genetic disorders and consequently as a substrate for drug discovery based on phenotypic reversal. Neuruloids modified to carry the HTT gene with expanded CAG repeats (e.g., 43, 48, 56, 65, 72, and 150 repeats) reflect a diversity of poly-Q lengths as observed in patients suffering from HD and are characterized with a HD phenotype that includes an expansion of the PAX6 neural rosette (Harekami et al., 2019, Nature Biotechnology 37:1198-1208). The BRD9 inhibitors can act on the neuruloids to reverse the expansion of the PAX6 expressing neural rosette induced by CAG expansion. The BRD9 inhibitors for use in the methods the disclosure preferably partially or completely reverse the HD neuruloid phenotype with an EC₅₀ of less than 1 μM. In particular embodiments, the BRD9 inhibitors of the disclosure partially or completely reverse the HD phenotype with an EC₅₀ of less than 750 nM, less than 500 nM, less than 300 nM or less than 200 nM.

In some embodiments, wild type (WT) neuruloids remain unaffected at the EC₅₀ for HD phenotype reversal, indicating the lack of pleiotropic effects that could be reflective of toxicity at a therapeutic dose. In certain aspects, the BRD9 inhibitors for use in the methods of the disclosure have an EC₅₀ for reversing the HD neuruloid phenotype that is at least 5 times lower than the EC₅₀ for inducing pleiotropic effects in WT neuruloids. In particular embodiments, the EC₅₀ for reversing the HD neuruloid phenotype is at least 10 times lower, at least 20 times lower or at least 50 times lower than the EC₅₀ for inducing pleiotropic effects in WT neuruloids.

The BRD9 inhibitors are typically administered in the form of a pharmaceutical composition together with one or more adjuvants, excipients, carriers, buffers, diluents, and/or other customary pharmaceutical auxiliaries. Further details on techniques for formulation and administration may be found in the latest edition of Remington's Pharmaceutical Sciences (Maack Publishing Co., Easton, Pa.).

The BRD9 inhibitors can be formulated or co-administered with agents to improve delivery across the blood-brain barrier (“BBB”), for example as described in Pardridge et al., 2007, Drug. Discov. Today 12(1-2):54-61.

In certain embodiments, the BRD9 inhibitor is formulated or co-administered with an exosome or a carbon nanotube.

In certain embodiments, the BRD9 inhibitor is formulated or co-administered with a brain permeability enhancer, such as cereport, regadenoson, or borneol. Brain permeability enhancers such as cereport bind to the receptors on the surface of endothelial cells and kicks off a biochemical cascade that loosens the tight junctions.

The BRD9 inhibitors can also be administered in the form of amino acid conjugates, for example a lysine conjugate or a phenylalanine conjugate.

Other agents that improve delivery across the BBB are transporter peptides and proteins that allow delivery of specific molecules without disrupting the BBB. A peptidomimetic mAb, such as against the transferrin receptor can be used as a molecular “Trojan horse” to ferry any attached drug or gene across the BBB.

The BRD9 inhibitors can be encapsulated within nanoparticles, for examples nanoparticles that are lipid-based, albumin-based, apolipoprotein-based, polymer-based nanoparticle, dendrimer-based nanoparticle, or inorganic-based nanoparticle.

The administration of the BRD9 inhibitors can be accompanied by physical or electrical methods, for example microbubble-enhanced ultrasound or transcranial magnetic stimulation, that improve uptake through the BBB.

An effective amount of the BRD9 inhibitor to be administered is dependent upon many factors, including but not limited to, the type of disease or condition giving rise to an anticipated cerebral ischemic episode, the patient's general health, size, age, and the nature of treatment, i.e., short-term of chronic treatment. Generally, the treatment may be given in a single dose or multiple administrations, i.e., once, twice, three or more times, per day. The administrations can be given for a period of time ranging from one week or one month to chronically over an extended period of time.

In certain embodiments, a BRD9 inhibitor described herein can be used in the manufacture of a medicament for the treatment of Huntington's Disease.

### 7. EXAMPLES

### 7.1. Example 1: Induction and Analysis of Micro-Pattern Based Neuruloids

Gene expression within neuruloids was analyzed and compared with equivalent expression within the in vivo ectodermal compartment at neurulation stages.

7.1.1. Materials and Methods

Micropatterned cell culture on chips and neuruloid induction: Micropatterned glass coverslips (CYTOOCHIPS Arena A, Arena 500A, Arena EMB A) were first coated with 10 μg ml⁻¹ recombinant Laminin-521 (BioLamina, LN521-03) diluted in PBS+/+(Gibco) for 3 h at 37° C. Micropatterns were placed face-up onto a Parafilm that was seated in a 10 cm dish, then 800 μl of laminin solution was added to the micropattern. After 3 h at 37° C., the coated micropattern was transferred to a 35 mm dish with 5 ml of PBS+/+. Laminin was removed with six serial dilutions in PBS+/+(dilution 1:4) before two complete washes in PBS+/+. Then the coated micropattern was kept in PBS+/+ at 37° C. Cells were seeded as follows: cells growing in MEF-CM on culture dish were washed once with PBS−/− (Gibco), then treated with accutase (Stem Cell Technologies) for 5 min. Cells were then triturated with a pipette to ensure single cell suspension and accutase was diluted out with 4× HUESM medium supplemented with 20 ng ml⁻¹ bFGF and ROCK inhibitor, Y27632 (10 μM; Abcam ab120129). Cells were further diluted with the same medium and 5×10⁵ (or as indicated) cells in 3.0 ml of medium were placed over the micropattern in a 35-mm tissue culture dish, then incubated at 37° C. After 3 h, the micropattern in a dish was washed once with PBS+/+. For SB+LDN condition: PBS+/+ was replaced with 3 N medium73 with 10 μM SB431542 (Stemgent 04-0010-10) and 0.2 μM LDN 193189 (Stemgent 04-0019). At days 3 and 5, the medium was replaced with the same fresh medium and incubated at 37° C. until day 7. For SB+BM P4 condition (neuruloid), PBS was replaced with HUESM with 10 μM SB431542 and 0.2 μM LDN 193189. At day 3, the medium was replaced with HUESM with 10 μM SB431542 and BM P4 (50 ng ml⁻¹ or indicated each experiment). At day 5, the medium was replaced with the same fresh medium then incubated until day 7.

Immunofluorescence: Micropattern coverslips were fixed with 4% paraformaldehyde (Electron Microscopy Sciences 15713) in warm medium for 30 min, rinsed three times with PBS−/−, and then blocked and permeabilized with 3% normal donkey serum (Jackson Immunoresearch 017-000-121) with 2% Triton X-100 (Sigma 93443) in PBS−/− for 30 min. Micropatterns were incubated with primary antibodies for 1.5 h, washed three times in PBS−/− for 5 min each, incubated with secondary antibodies conjugated with Alexa 488, Alexa 555, Alexa 594 or Alexa 647 (1:1,000 dilution, Molecular Probes) and 10 ng ml−1 of DAPI (Thermo Fisher Scientific D1306) for 30 min and then washed two times with PBS−/−. For double staining with antibodies from the same species, Alexa 488 Fab fragments (Jackson Immunoresearch, 715-547-003) and Fab fragment IgG (Jackson Immunoresearch, 715-007-003) were used. Coverslips were mounted on slides using ProLong Gold antifade mounting medium (Molecular Probes P36934).

Microscopy: Micropattern coverslips were acquired on a Zeiss Inverted LSM 780 laser scanning confocal microscope with a ×10, ×20 or ×40 water-immersion objective.

7.1.2. Results

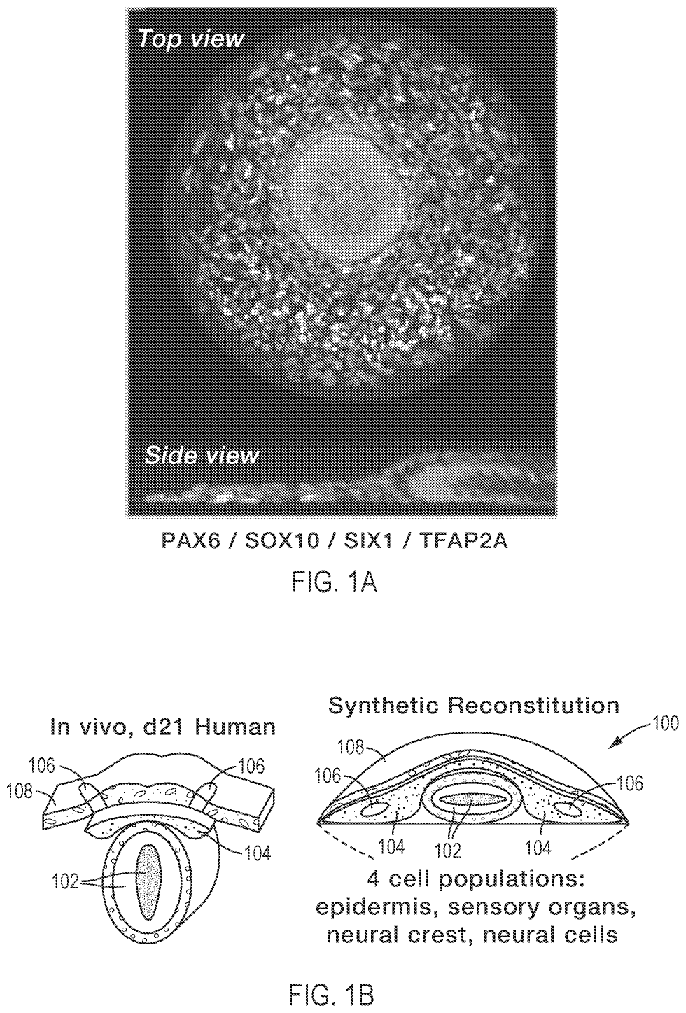

The comparison of the in vitro neuruloid (FIG. 1A) with the in vivo counterpart around day 21 post-fertilization (FIG. 1B) reveals a high level of similarities, making them an ideal pre-clinical endpoint for the study of human genetic disorders and screening for drugs based on phenotypic reversal.

### 7.2. Example 2: Characterization of Neuruloids Derived from HD Cell Lines

A library of eight HD RUES2 isogenic cell lines were induced to form neuruloids and screened for expression of a number of molecular markers.

7.2.1. Materials and Methods

Cell culture: All hESC lines were grown in HUESM medium that was conditioned with mouse embryonic fibroblasts and supplemented with 20 ng ml⁻¹ βFGF (MEF-CM)27. Cells were tested for Mycoplasma spp. at 2-monthly intervals. Cells were grown on tissue culture dishes coated with Geltrex (Life Technologies) solution and analyzed for the expression of various markers.

7.2.2. Results

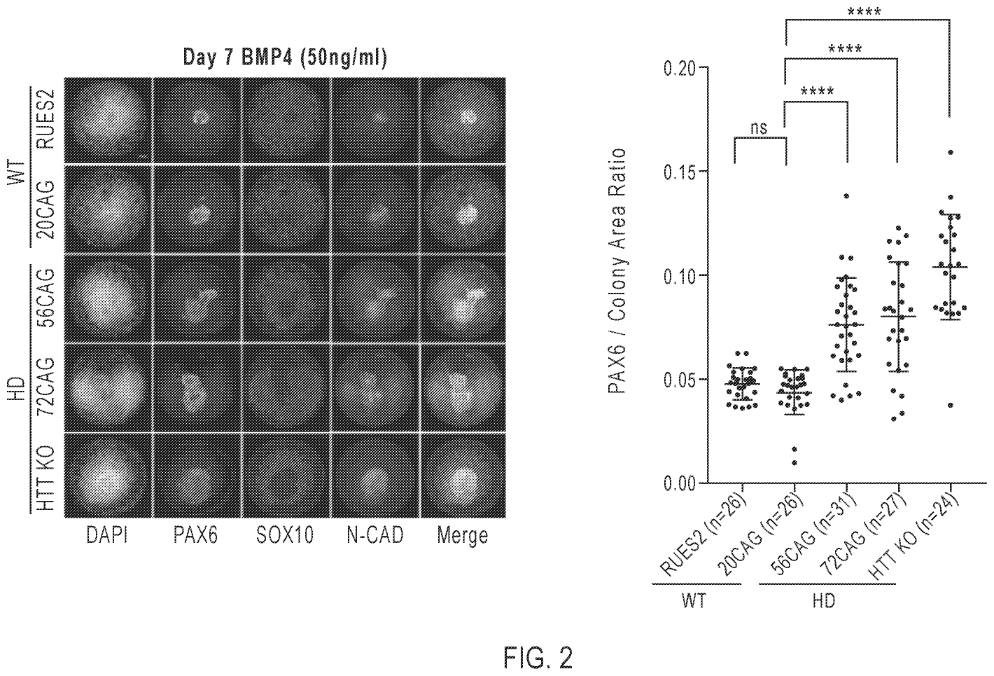

The neuruloids showed a clear feature: PAX6 area/rosette extension. In the neuruloids, CAG extension was associated with increased PAX6+ area (FIG. 2). The discovery of this CAG-expansion specific phenotype opens the possibility to use this phenotypic signature to perform high-throughput screening campaigns towards the discovery of small molecules that can reverse the HD phenotypes back to WT.

### 7.3. Example 3: An AI Screen for HD Phenotype Reversal

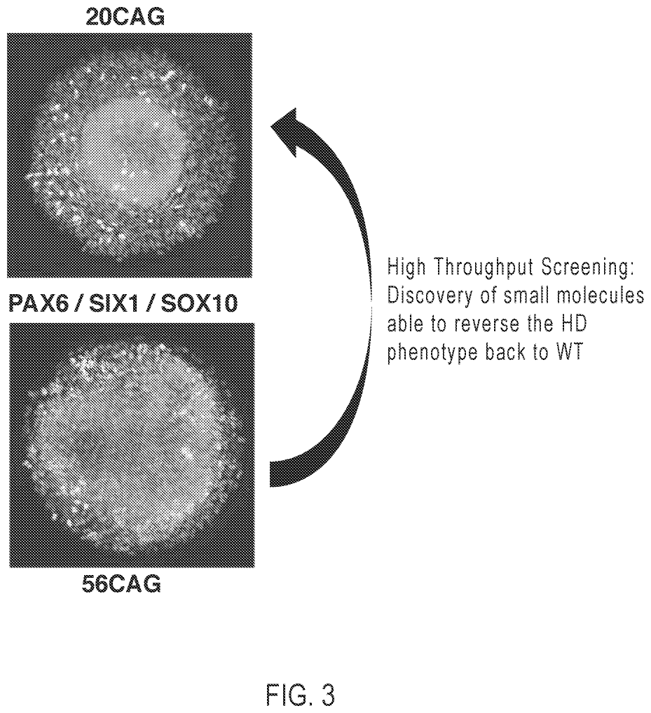

An AI screen was performed for molecules that could reverse the HD neuruloid phenotype to wildtype (see FIG. 3). In such a screen, molecules able to act at the colony level to reverse the deleterious effects induced by CAG expansion while leaving WT colonies unaffected will be specific to the disease allele and therefore considered as promising therapeutic candidates that will progress towards in vivo validation in animal models of HD.

The easiest feature that could be used to characterize neuruloid phenotypes is PAX6+ domain extension. This represents a useful specific feature but does not encompass the full scope of phenotypic variations likely to arise from a large phenotypic screen. Ideally, an analytical tool is needed to allow 1) strong discrimination between the WT and HD neuruloids in order to minimize the number of false positives/false negatives; 2) quantitative measurement of the degrees of phenotypic reversal by drawing a scale between the WT and HD phenotypes; 3) measurement of the cytotoxicity and the off-target effects of the small molecules tested.

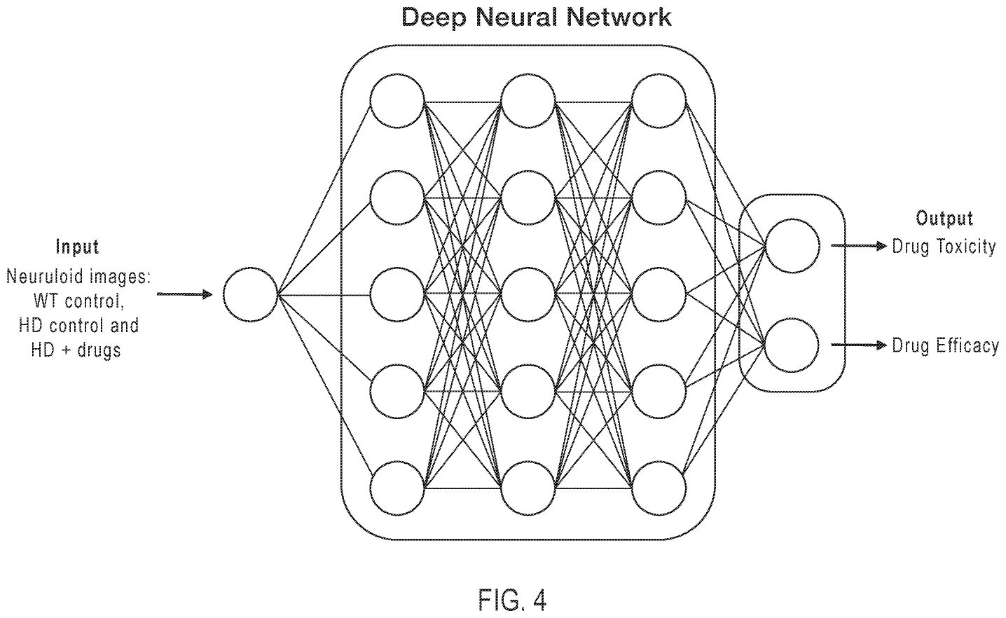

Over the last years, machine learning tools based on deep neural networks have been used to perform similar tasks for facial recognition in security videos or for search engines. These tools can also be used for drug discovery. Specifically, the screening campaigns return large number of images of neuruloids: WT control, HD control, and HD treated with drugs. All these images can be used as the input of a deep neural network specifically trained to return two quantities that are predictive of a drug success in the clinic: drug toxicity and drug efficacy (FIG. 4).

7.3.1. Materials and Methods

Micropatterned cell culture on 96 well plates and imaging: The micropatterned cell culture on chips and neuruloid induction protocol was used with micropatterned 96 well plates (CYTOOPLATES Arena A, Arena 700). The neuruloid induction was modified so that all steps requiring media changes, cell dispensing as well as washes and incubations for immunofluorescence were performed with a EL406 washer/dispense robot. For cell seeding, a volume of 200 μl cell suspension at a density of 0.18M cells per ml was used. Plate imaging was performed with an InCell Analyzer 2000 high-content imager through a 4× lens.

A number of libraries consisting of 2080 compounds were screened in 96 well plates with micropatterned glass bottoms. In these plates, each well has around 27 individual neuruloids. During the screen, two plates of WT controls and two plates of HD untreated controls were reserved in order to have enough control data for training the deep neural network for later analysis. Compounds were applied to the test plates at day 0 of differentiation and re-applied at day 3 and day 5 during media change steps. Each compound was applied in a unique well, at a unique concentration of 10 uM. At day 7, plates were fixed and stained for DAPI, PAX6 and Phalloidin. After staining, plates were imaged and individual neuruloids in every well were segmented and labeled before being fed to the specific deep neural network.

Image analysis: image tiles acquired from micropatterned or plate culture experiments were stitched and background-corrected. A foreground mask was created to detect colonies by thresholding the DAPI channel and calculating alpha shapes in respect to colony size. Each detected colony was extracted from the corrected and stitched image.

Deep learning for quantification of phenotypic rescue: Multichannel WT and disease organoid images are split into training (70%) and validation (30%) images. A neural network (NN) is then trained on the training data set. The NN is coded using a machine-learning framework such as Pytorch. For 2D images, this framework provides convolutional NNs pre-trained on the ImageNet database. Residual Networks (ResNets) are a subclass of convolutional networks and are particularly efficient at classifying images. Pre-trained ResNets of different depths (with 18, 34, 50, 101 or 152 layers) are available in all major machine-learning frameworks. ResNet50 was selected. It consists of blocks of layers made up of convolutional, Batch Normalization (BatchNorm) and Rectified Linear (ReLU) layers. A final Average Pooling and densely connected layer are removed and replaced by custom layers. First, the pre-trained network classifies images into many more classes than WT and disease. Also, the last layers of a NN are more specific to the dataset than the initial layers. Therefore, the last Average Pooling and fully connected layer are removed and replaced with untrained Adaptive Average Pooling, Adaptive Maximum Pooling, Batch Norm, Dropout and fully connected layers, followed by a final softmax operation. Here, a fully connected layer of 512 units is used and directly afterwards the final fully-connected layer consists of only two units, one each for WT and disease. A final softmax converts the activation of these units into probabilities that sum to 1.

Training is performed by showing images to the network, comparing the output probabilities of WT or disease to the true value, and changing the network weights such that the next time the image is shown the network would give a prediction that is closer to the true value. This fitting procedure is performed using the backpropagation algorithm, which is implemented in all major neural network frameworks. The images are shown to the network many times (each run is called an “epoch”). Images are “augmented”, i.e. a set of image transformation is applied to them that does not significantly change the content of the image but enlarges the pool of images that the network can learn from. These data augmentation operations consist of rotations, cropping, scaling the image from 90-110%, and changing the contrast of the images. Training is done several times with different hyperparameters (number of layers, momentum and learning rate, dropout percentage, number of epochs) to find an optimal set of these parameters.

Analysis of the images from the screen is then carried out using the network trained on the training images. First, the accuracy of the network is verified by using untreated control organoids from the screen. Then, images from organoids treated with drug compounds are analyzed by the network that assigns a score between 0 and 1 to each image, where 1 represents WT and 0 represents disease. In the analysis of the screen, wells with less than 10 intact organoids were excluded from the analysis, assuming that in those cases the compound had a toxic effect. For the others, the network measured, for each well, the capacity of compounds to reverse phenotypes based on the tools described in this section and this score was averaged across each well.

Deep learning for quantification of cytotoxicity: An autoencoder based method was used to asses toxicity. These unsupervised neural networks encode data and compress it in a low dimensional latent representation. The unsupervised nature of this machine learning method has the advantage that the autoencoder learns a representation of the data without any additional information about the data (such as that it is derived from wild type or disease cell lines), and is thus unbiased in estimating the toxicity of compounds. The representation of the data in terms of vectors also has the advantage that differences in the wild type and disease phenotype can be removed from the vector space, since this difference is not relevant in determining toxicity. Toxicity was determined in the following way: First, the difference between wild type and disease is removed from the latent space. Then, the distance from the mean vector of the wild type and disease phenotypes is calculated, and compared to the standard deviation of the wild type and disease phenotypes. This distance is defined as the toxicity.

7.3.2. Results

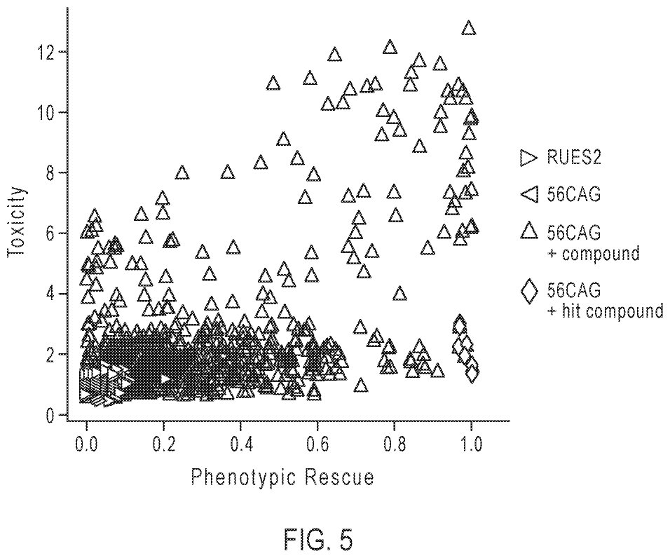

Analysis results are shown in FIG. 5. Most compounds fall in the bottom left quadrant with molecules basically not affecting the HD neuruloids. A number of molecules had a toxic signature (toxicity score >3) and a handful of hits were falling in the hit space defined by the characterization of the AI analytical tool, defined as high phenotypic rescue score (>0.95) and low toxicity (<3). In particular, Bromosporine was discovered to be a particularly effective molecule with low toxicity (phenotypic rescue=0.98 and toxicity score=1.93).

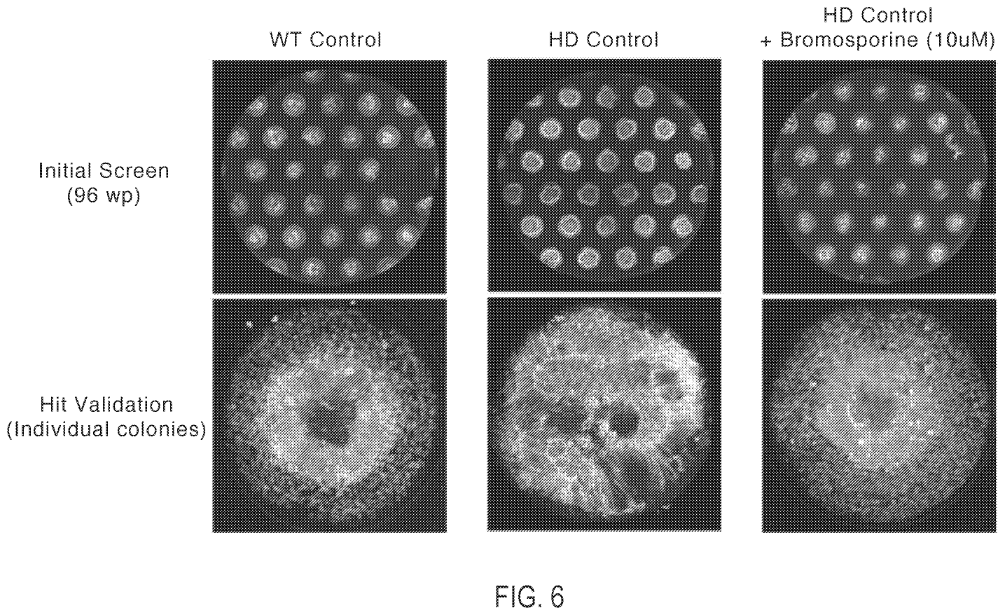

FIG. 6 (top) shows examples of the images acquired in the primary screen with a WT control well, a HD control well and the HD well treated with 10 μM Bromosporine. This qualitatively shows enlarged PAX6 area in the HD background and its reduction towards WT level with Bromosporine, but also a reversal of the HD phenotype towards the WT configuration when the HD neuruloids are in contact with Bromosporine, which match the quantitative results obtained from the AI algorithm and presented in FIG. 5.

Bromosporine is described in the literature as a broad-spectrum inhibitor for BRDs with IC₅₀ of 0.41 μM, 0.29 μM, 0.122 μM and 0.017 μM for BRD2, BRD4, BRD9 and CECR2, respectively. In order to confirm Bromosporine as a modulator of HD phenotypes in vitro, Bromposporine reconstituted from fresh stock power to validate its properties in a low scale experiment. A qualitative result is shown in FIG. 6 (bottom), which clearly shows the rescue of the disorganization induced by the HD gene by application of 0.5 μM Bromosporine.

### 7.4. Example 4: Quantification of Bromosporine Potency and Toxicity

In order to measure quantitatively the potency of Bromosporine at reversing HD neuruloids phenotypes, a dose response experiment was conducted.

7.4.1. Materials and Methods

HD neuruloids were contacted with 10 different concentration of Bromosporine in triplicates. The degree of phenotypic reversal was quantified using the AI algorithm for each Bromosporine concentration.

7.4.2. Results

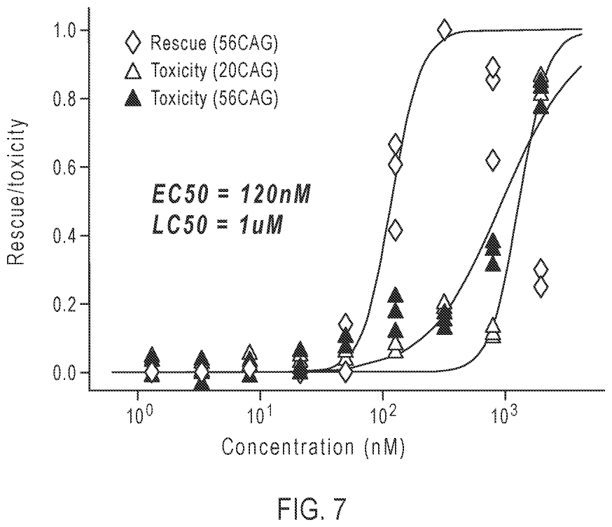

Bromosporine was fully effective at around 300 nM with an EC₅₀ of 120 nM (FIG. 7). The toxic profile of Bromosporine was assessed on WT and HD-56CAG neuruloids. A toxic response started to occur at concentrations above 1 μM. Overall, this shows that Bromosporine is efficient in a complex in-vitro model of HD and that there is a window of concentration, between 0.3 μM and 1 μM, in which Bromosporine does not affect WT neuruloids but can rescue the HD neuruloids. This highlights a window of specificity for the disease allele in this assay system. Moreover, in this concentration range, Bromosporine shows low toxicity.

### 7.5. Example 5: Assaying BRD Inhibitors for HD Neuruloid Phenotype Reversal

An assay was performed to discover whether other BRD inhibitors had HD neuruloid phenotype reversing activity.

7.5.1. Materials and Methods

A selection of BRD inhibitors were tested using 96 well plates. The efficacy and toxicity of each compound was determined.

7.5.2. Results

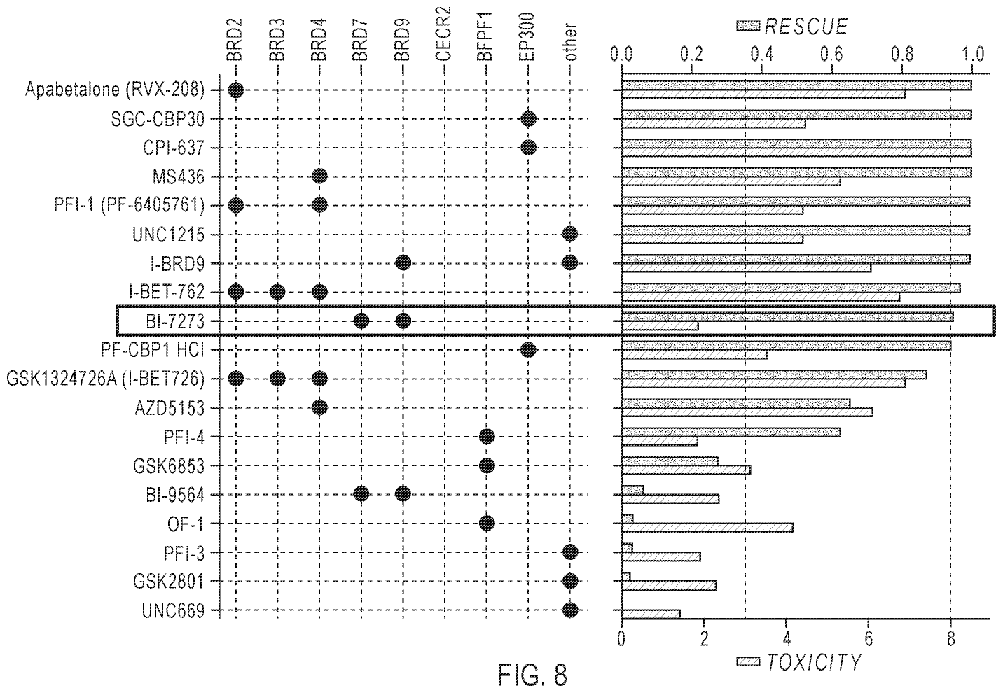

A selection of 19 BRD inhibitors were tested at a single concentration of 10 μM using 96 well plates. Efficacy and toxicity are shown plotted in FIG. 8 together with the target of each molecule. Using the same quantitative criteria as the ones used for hit definition during the primary screen, only one molecule showed high enough efficacy while keeping a low toxicity: B17273. This molecule is known to be a potent, selective, and cell-permeable BRD9 inhibitor with IC₅₀s of 19 nM and 117 nM for BRD9 and BRD7 respectively.

The fact that B17273 also scores positively reinforces the finding that BRD inhibitors are efficient in an in vitro model of HD. Moreover, as BRD9 is the only common denominator between the molecular targets of Bromosporine and B17273, it raises the possibility that BRD9 is actually the relevant target in the present assay system that might be inhibited to rescue HD phenotypes.

While other BRD9 inhibitors such as B19564 did not score positively in the experiment with the panel of BRD inhibitors (FIG. 8), the testing of these molecules at a single and rather high concentration of 10 μM could potentially hinder the activity of highly potent molecules that can start to present toxic and off-target effects at high concentrations.

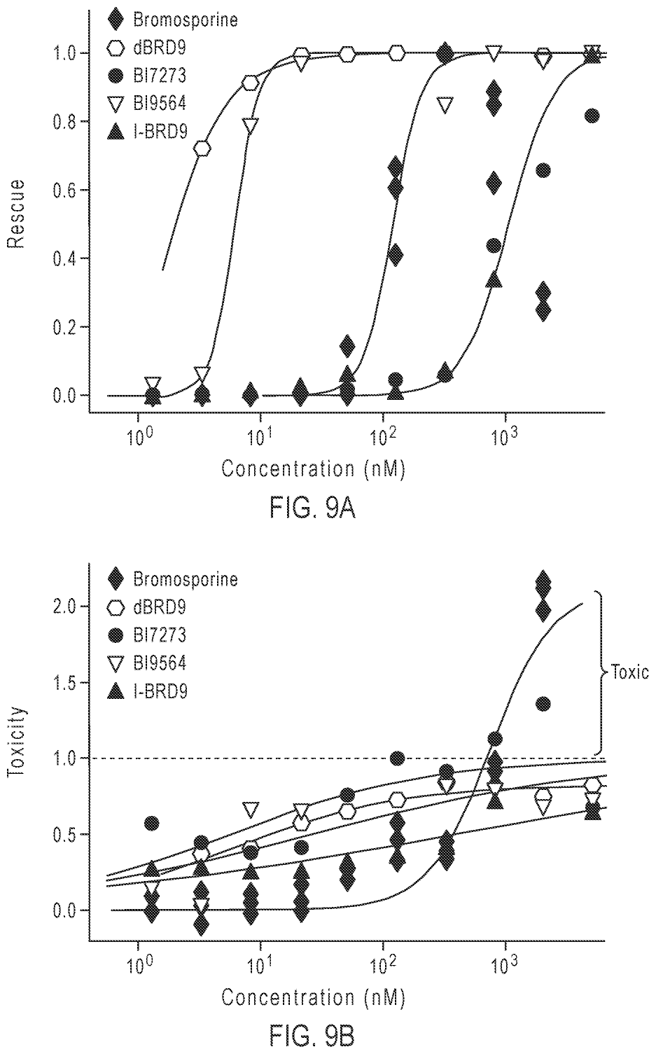

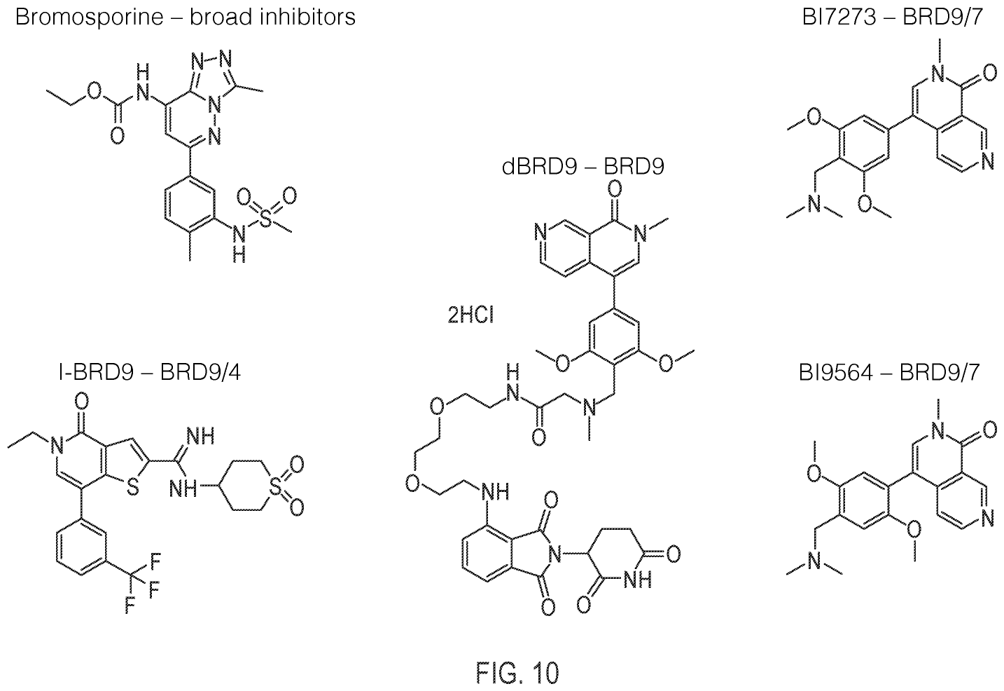

Following this hypothesis, the concentration dependent response of other BRD9-focused inhibitors were tested at lower concentrations. These consisted of Bromosporine (targets: BRD2, BRD4, BRD9 and CECR2) and B17273 (targets: BRD9 and BRD7) as positive controls, but also BI9564 (targets: BRD9 and BRD7), dBRD9 (selective BRD9 protac) and I-BRD9 (BRD9 and BRD4). Results are presented in FIG. 9. Strikingly, all of these molecules were potent in rescuing the HD neuruloids with sub-micromolar activity (FIG. 9a). Moreover, toxic profiles of BI7273, BI9564, d-BRD9 and I-BRD9 were all lower than Bromosporine (FIG. 9b). This might suggest that the broad activity of Bromosporine can become detrimental at high concentrations compared to more BRD9-centric compounds. Successful BRD9 inhibiting compounds together with a brief description of their activities are shown in FIG. 10.

### 7.6. Example 6: Mechanism of Action of BRD Inhibitors

In order to decipher the mechanism of action of BRD inhibitors, BRD inhibitors were tested for HTT lowering abilities based in part on the fact that an HTT lowering strategy has shown efficacy in an HD mice model and is the basis of the latest clinical developments for HD. HTT lowering is therefore the only known mechanism of action for hoping to treat HD.

We therefore repeated our protocol for the formation of HD neuruloids in two genetic backgrounds: 56CAG and 72CAG and treated with the 5 BRD inhibitors disclosed in FIG. 9 at two concentrations: 1 uM and 5 uM. At the end of the differentiation protocol, neuruloids lysates were frozen and later analyzed for their HTT contents. Both expanded HTT levels and total HTT levels (expanded+regular) were quantified through ELISA-based meso scale discovery (MSD) electrochemiluminescence assay.

7.6.1. Materials and Methods

HD neuruloids were created in two genetic backgrounds: 56CAG and 72CAG; and treated with the 5 BRD inhibitors disclosed in FIG. 10 at two concentrations: 1 μM and 5 μM. At the end of the differentiation protocol, neuruloid lysates were analyzed for their HTT contents. Both expanded HTT levels and total HTT levels (expanded+regular) were quantified through ELISA-based meso scale discovery (MSD) electrochemiluminescence assay.

7.6.2. Results

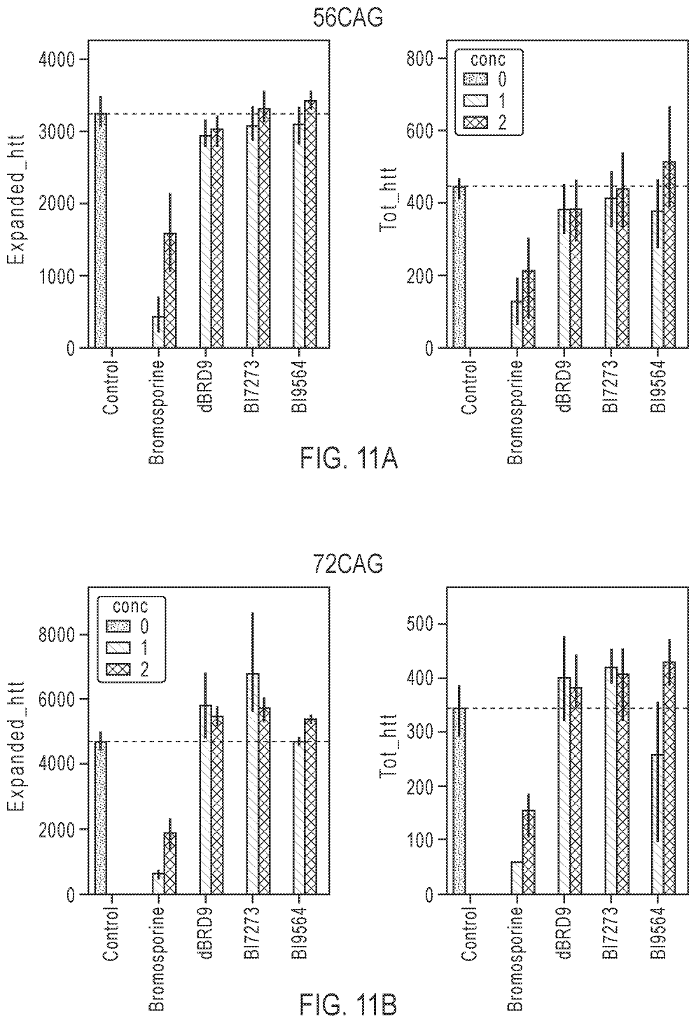

The results are presented in FIG. 11. Among the 4 drugs tested, bromosporine, B17273, B19564 and dBRD9, only Bromosporine had HTT levels reduction abilities. Specifically, Bromosporine reduced both expanded HTT and total HTT levels at the two concentrations tested and in the two genetic backgrounds of 56CAG and 72CAG. Reductions of HTT levels were significant with more than 75% reduction of expanded HTT levels in all backgrounds at 5 μM and more than 50% at a lower concentration of 1 μM. Interestingly, no HTT reduction ability was measured with B17273, B19564 and dBRD9. This suggests that these three molecules act through a different mechanism of action than HTT level reductions to rescue the HD neuruloids.

### 7.7. Example 7: BRD Inhibitor-Mediated Rescue of an HD Phenotype in Human Gastruloids

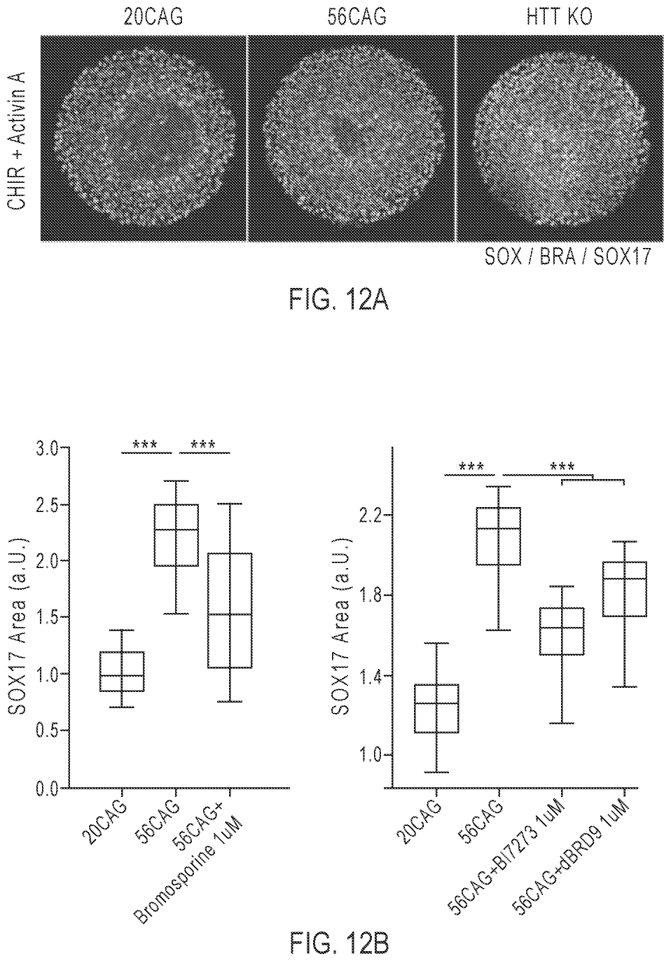

BRD inhibitors were tested to determine whether they were able to rescue another HD phenotype in human gastruloids. When human embryonic stem cell colonies are stimulated for 48 hrs with CHIR and Activin a primitive-streak like population, SOX17+, is induced at the periphery (see FIG. 12a). Moreover, in HD gastruloids made from the HD-56CAG cells, the SOX17+ domain at the periphery is greatly expanded. This effect is even more pronounced when using a null HTT−/− cell line, which supports the findings that the HD mutation is in fact a loss of function mutation in a developmental context, as it phenocopies the absence of the protein.

7.7.1. Materials and Methods

Gastruloid induction: Using the protocol for coating micropatterned chips. after the final wash in PBS+/+, 8×10⁵ cells were seeded on each coverslip in a defined volume of MEF-CM supplemented with 20 ng/ml bFGF (R&D Systems), 10 μM ROCK inhibitor (Y-27632, Abcam), 1× Penicillin-streptomycin (Thermo Fisher Scientific), 100 μg/ml Normocin (Invivogen) and left unperturbed for 10 minutes to ensure homogenous distribution across the patterns. ROCK inhibitor was removed from the medium 3 hours after seeding and cells were induced the following day with 50 ng/ml BM P4 (R&D Systems), 2 μM IWP2 (Stemgent), 6 μM CHIR99021 (EMD Millipore), 100 ng/ml ACTIVIN (R&D Systems) and small molecule treatments. Samples were fixed 48 hours later and analyzed by immunofluorescence.

7.7.2. Results

When 1 μM Bromosporine, BI7273 or dBRD9 were applied to the HD gastruloids, in each case there was a significant reduction of the SOX17+ area at the periphery, towards the WT configuration (see FIG. 12b). This clearly shows that in a second HD phenotype, orthogonal to the neuruloids, BRD inhibitors maintain their ability to perform phenotypic rescue.

### 7.8. Example 8: Reducing BRD9 Levels Using an Inducible CRISPR/Cas System Rescues HD Phenotype

7.8.1. Materials and Methods

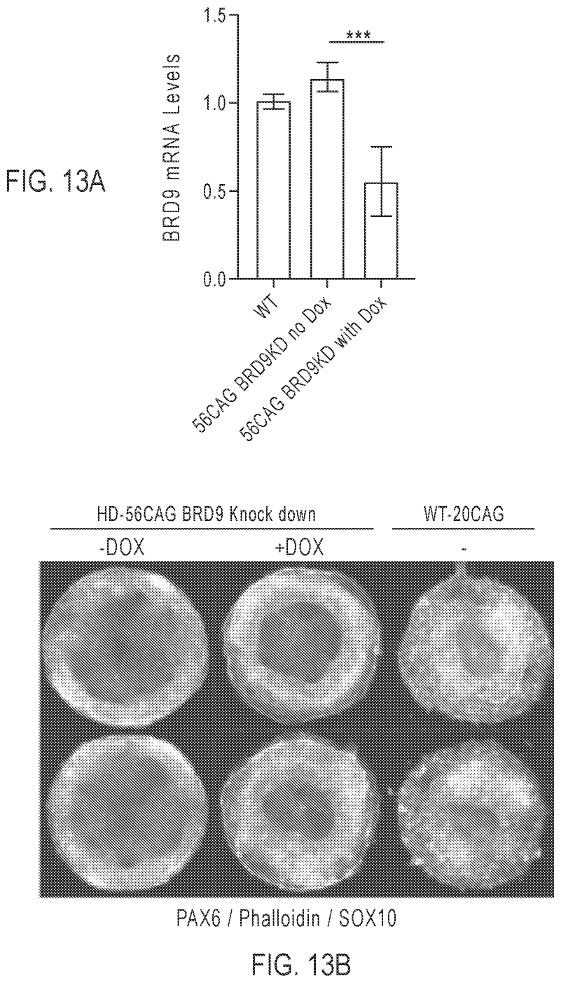

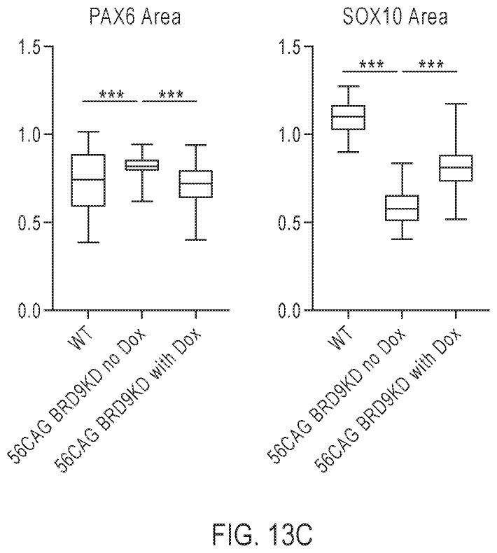

An inducible BRD9 knockdown line was generated in the HG-56CAG background that can efficiently lower BRD9 transcripts. Utilizing a doxycycline-induced Tet-based CRISPR/Cas9 system, BRD9 transcripts were lowered following addition of doxycycline (DOX). Optimization of three individual gRNAs resulted in a construct capable of lowering BRD9 mRNA levels by 50% following 2 days of DOX application (FIG. 13A). The HD phenotype was analyzed and compared in the inducible knockdown line and WT-20CAG neuruloids which were used as a control.

7.8.2. Results

Analysis of the HD-56CAG and WT-20CAG neuruloids revealed a disorganization of the central PAX6+ domain that became extended, and a reduction of the peripheral SOX10+ cells in HD-56CAG relative to control (FIGS. 13B and 13C). Upon application of DOX and lowering BRD9 levels in the 56CAG background, a significant rescue in these two HD-associated parameters towards their WT values was observed (FIGS. 13B and 13C). These data confirm that BRD9 is HD target of interest and that its inhibition leads to reversal of HD phenotypes.

### 8. SPECIFIC EMBODIMENTS

While various specific embodiments have been illustrated and described, it will be appreciated that various changes can be made without departing from the spirit and scope of the disclosure(s). The present disclosure is exemplified by the numbered embodiments set forth below.

1. A method of treating a subject having Huntington's Disease (HD), comprising administering to the subject a therapeutically effective amount of a bromodomain 9 (BRD9) inhibitor.

2. The method of embodiment 1, wherein the BRD9 inhibitor is not conjugated to a degron.

3. The method of embodiment 1, wherein the administration of the BRD9 inhibitor does not induce proteasome-mediated degradation of BRD9 in vivo.

4. The method of any one of embodiments 1 to 3, wherein the BRD9 inhibitor is a selective BRD9 inhibitor.

5. The method of embodiment 4, wherein the BRD9 inhibitor has at least 2-fold or at least 5-fold greater inhibition of BRD9 as compared to BRD2.

6. The method of embodiment 4 or embodiment 5, wherein the BRD9 inhibitor has at least 2-fold or at least 5-fold greater inhibition of BRD9 as compared to BRD3.

7. The method of any one of embodiments 4 to 6, wherein the BRD9 inhibitor has at least 2-fold or at least 5-fold greater inhibition of BRD9 as compared to BRD4.

8. The method of any one of embodiments 1 to 7, wherein the BRD9 inhibitor is a pyridinone compound.

9. The method of any one of embodiments 1 to 7, wherein the BRD9 inhibitor is BI-9564:

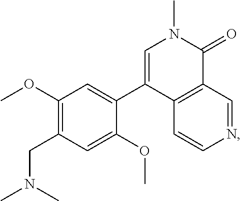 [SMILES: COc1cc(-c2cn(C)c(=O)c3cnccc23)c(OC)cc1CN(C)C]

or a pharmaceutically acceptable salt thereof.

10. The method of any one of embodiments 1 to 7, wherein the BRD9 inhibitor is BI-7273:

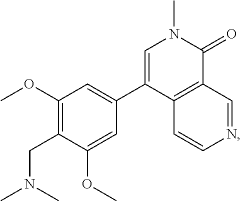 [SMILES: COc1cc(-c2cn(C)c(=O)c3cnccc23)cc(OC)c1CN(C)C]

or a pharmaceutically acceptable salt thereof.

11. The method of any one of embodiments 1 to 7, wherein the BRD9 inhibitor is a methylquinolinone compound.

12. The method of any one of embodiments 1 to 7, wherein the BRD9 inhibitor is LP-99:

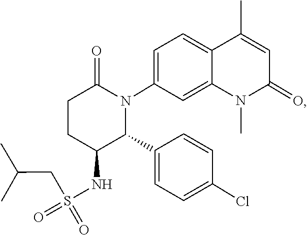 [SMILES: Cc1cc(=O)n(C)c2cc(N3C(=O)CC[C@H](NS(=O)(=O)CC(C)C)[C@H]3c3ccc(Cl)cc3)ccc12]

or a pharmaceutically acceptable salt thereof.

13. The method of any one of embodiments 1 to 7, wherein the BRD9 inhibitor is a thienopyridone compound.

14. The method of any one of embodiments 1 to 7, wherein the BRD9 inhibitor is I-BRD9:

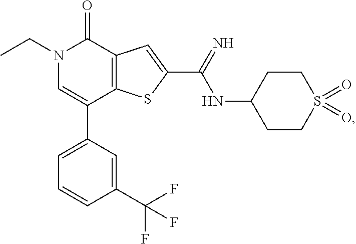 [SMILES: CCn1cc(-c2cccc(C(F)(F)F)c2)c2sc(C(=N)NC3CCS(=O)(=O)CC3)cc2c1=O]

or a pharmaceutically acceptable salt thereof.

15. The method of any one of embodiments 1 to 7, wherein the BRD9 inhibitor is d-BRD9:

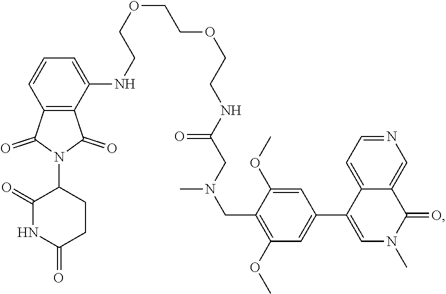 [SMILES: COc1cc(-c2cn(C)c(=O)c3cnccc23)cc(OC)c1CN(C)CC(=O)NCCOCCOCCNc1cccc2c1C(=O)N(C1CCC(=O)NC1=O)C2=O]

or a pharmaceutically acceptable salt thereof.

16. The method of any one of embodiments 1 to 7, wherein the BRD9 inhibitor is TP-472:

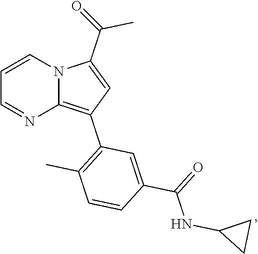 [SMILES: CC(=O)c1cc(-c2cc(C(=O)NC3CC3)ccc2C)c2ncccn12]

or a pharmaceutically acceptable salt thereof.

17. The method of any one of embodiments 1 to 7, wherein the BRD9 inhibitor is GNE-375:

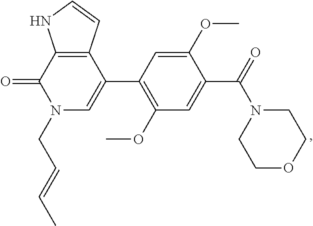 [SMILES: C/C=C/Cn1cc(-c2cc(OC)c(C(=O)N3CCOCC3)cc2OC)c2cc[nH]c2c1=O]

or a pharmaceutically acceptable salt thereof.

18. The method of any one of embodiments 1 to 7, wherein the BRD9 inhibitor is a compound of Formula (I):

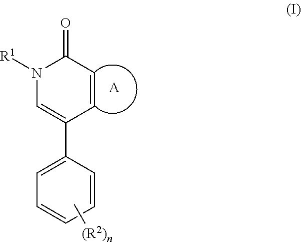

or an enantiomer, diastereomer, stereoisomer, or pharmaceutically acceptable salt thereof, wherein:
A is phenyl or 5- or 6-membered heteroaryl containing 1 or 2 heteroatoms selected from N and S, wherein the phenyl or heteroaryl is unsubstituted or substituted with 1 to 3 R³ groups;
R¹ is H, (C₁-C₄)alkyl, or (C₁-C₄)haloalkyl;
each R² is independently (C₁-C₄)alkyl, (C₁-C₄)haloalkyl, (C₁-C₄)alkoxy, (C₁-C₄)haloalkoxy, halogen, OH, or NH₂;
each R³ is independently (C₁-C₄)alkyl, (C₁-C₄)haloalkyl, (C₁-C₄)alkoxy, (C₁-C₄)haloalkoxy, halogen, OH, NH₂, or

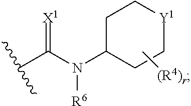

X₁ is NR⁵ or O;
Y₁ is S(O)ₐ or NR⁵;
each R⁴ is independently (C₁-C₄)alkyl, (C₁-C₄)haloalkyl, halogen, or —C(O)(C₁-C₃)alkyl;
each R⁵ is independently H or (C₁-C₄)alkyl;
each R⁶ is independently H or (C₁-C₄)alkyl;
a is 0, 1, or 2; and
n and r are each independently 0, 1, 2, or 3.

19. The method of any one of embodiments 1 to 7, wherein the BRD9 inhibitor is a compound of Formula (II):

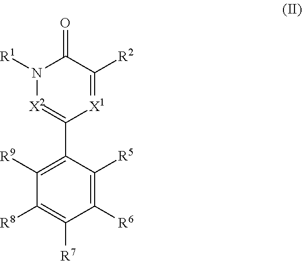

or an enantiomer, diastereomer, stereoisomer, or pharmaceutically acceptable salt thereof, wherein:
R¹ is (C₁-C₃)alkyl or cyclopropyl;
R² is halogen, (C₁-C₃)alkyl, (C₁-C₃)haloalkyl, NH₂, NH(C₁-C₃)alkyl or OH;
X¹ is N or CR³, and X² is N or CR⁴; provided that X¹ and X² cannot be both N;
R³ is H or (C₁-C₃)alkyl;
R⁴ is H or (C₁-C₃)alkyl; provided that R³ and R⁴ cannot be both (C₁-C₃)alkyl;
alternatively, R² and R³ taken together form a benzene ring or a 5-6 membered heteroarene ring, each of which rings can be independently unsubstituted or substituted with one or more groups which are independently halogen, OH, NH₂, NH(C₁-C₃)alkyl or (C₁-C₃)alkyl, wherein the (C₁-C₃)alkyl group can be unsubstituted or substituted with 5-6 membered heteroaryl or phenyl;
R⁵ and R⁹ are the same or different and are independently H, O(C₁-C₃)alkyl or (C₁-C₃)alkyl;
R⁶ and R⁸ are the same or different and are independently H, OH, halogen, NH₂, (C₁-C₃)alkyl, O(C₁-C₃)alkyl, O(C₁-C₃) haloalkyl, (C₁-C₃)alkyl-O—(C₁-C₃)alkyl, 4-7 membered heterocycloalkyl, (C₁-C₃)alkyl-SO₂—(C₁-C₃)alkyl, (C₁-C₃)alkyl-NH₂, (C₁-C₃)alkyl-N((C₁-C₃)alkyl)₂, N((C₁-C₃)alkyl)₂, or NHR¹³;
R¹³ is independently for each occurrence SO₂—(C₁-C₃)alkyl or (C₁-C₃)alkyl, wherein the (C₁-C₃)alkyl groups are unsubstituted or substituted with 5 to 6 membered heteroaryl;
alternatively, R⁵ and R⁶ taken together form a benzene ring;
alternatively, R⁷ and R⁶ or R⁷ and R⁸ taken together form a 5-7 membered heterocycloalkyl which is unsubstituted or substituted with (C₁-C₃)alkyl;
R⁷ is H, NH₂, Y—R¹², (C₁-C₃)alkyl or 4-7 membered heterocycloalkyl;
Y is CR¹⁰R¹¹, SO₂ or CO;
R¹⁰ and R¹¹ are the same or different and are independently H or (C₁-C₃)alkyl; or R¹⁰ and R¹¹ taken together form a C₃₋₄ cycloalkyl;
R¹² is NH₂, OH, (C₁-C₃)alkyl, N(R¹⁵,R¹⁶), OR¹⁷, aryl, or 5-6 membered heteroaryl, wherein the aryl or heteroaryl are independently unsubstituted or substituted with one or more halogen or 4-7 membered heterocycloalkyl, each of which heterocycloalkyl is independently unsubstituted or substituted with one or more groups selected from halogen, OH, NH₂, (C₁-C₃)alkyl, NH(C₁-C₃)alkyl, N((C₁-C₃)alkyl)₂, O(C₁-C₃)alkyl and CH₂R¹⁴;
R¹⁴ is 5-10 membered mono- or bicyclic aryl or heteroaryl, which is unsubstituted or substituted with NH₂, OH, halogen, CN, (C₁-C₃)alkyl, or O(C₁-C₃)alkyl;
R¹⁵ is H or (C₁-C₃)alkyl;
R¹⁶ is (C₁-C₃)alkyl, C₂₋₃ alkyl-N((C₁-C₃)alkyl)₂, C₂₋₃alkyl-NH(C₁-C₃)alkyl or 4-7 membered heterocycloalkyl, which heterocycloalkyl is unsubstituted or substituted with (C₁-C₃)alkyl;
R¹⁷ is (C₁-C₃)alkyl or 4-7 membered heterocycloalkyl, which heterocycloalkyl is unsubstituted or substituted with (C₁-C₃)alkyl;
wherein when R⁷ is YR¹², R⁶ and R⁸ can be the same or different and are independently H, OH, halogen, NH₂, CN, (C₁-C₃)alkyl, (C₁-C₃) haloalkyl, O(C₁-C₃)alkyl, O(C₁-C₃) haloalkyl or (C₁-C₃)alkyl-O—(C₁-C₃)alkyl; and
wherein at least one of the substituents R⁵ to R⁹ is not hydrogen.

20. The method of any one of embodiments 1 to 3, wherein the BRD9 inhibitor is a non-selective BRD9 inhibitor.

21. The method of embodiment 20, wherein the BRD9 inhibitor is bromosporine:

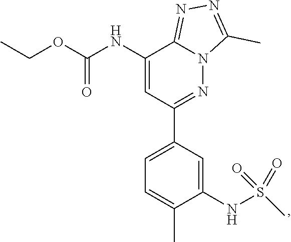 [SMILES: CCOC(=O)Nc1cc(-c2ccc(C)c(NS(C)(=O)=O)c2)nn2c(C)nnc12]

or a pharmaceutically acceptable salt thereof.

22. The method of any one of embodiments 1 to 21, wherein the BRD9 inhibitor is administered in the form of an amino acid conjugate.

23. The method of embodiment 22, wherein the amino acid conjugate is a lysine conjugate.

24. The method of embodiment 22, wherein the amino acid conjugate is a phenylalanine conjugate.

25. The method of any one of embodiments 1 to 24, wherein the BRD9 inhibitor is administered to the subject with a carrier.

26. The method of embodiment 25, wherein the carrier comprises a nanoparticle, an exosome, or a carbon nanotube.

27. The method of embodiment 26, wherein the carrier comprises a nanoparticle.

28. The method of embodiment 27, wherein the nanoparticle comprises a lipid-based nanoparticle.

29. The method of embodiment 27, wherein the nanoparticle comprises a human serum albumin-based nanoparticle.

30. The method of embodiment 27, wherein the nanoparticle comprises an apolipoprotein-based nanoparticle.

31. The method of embodiment 27, wherein the nanoparticle comprises a polymer-based nanoparticle.

32. The method of embodiment 27, wherein the nanoparticle comprises a dendrimer-based nanoparticle.

33. The method of embodiment 27, wherein the nanoparticle comprises an inorganic-based nanoparticle.

34. The method of embodiment 26, wherein the carrier comprises an exosome.

35. The method of embodiment 27, wherein the carrier comprises a carbon nanotube.

36. The method of any one of embodiments 1 to 35, further comprising adjunctively administering a brain permeability enhancer to the subject.

37. The method of embodiment 36, wherein the BRD9 inhibitor and the brain permeability enhancer are co-administered in a single pharmaceutical composition.

38. The method of embodiment 36 or embodiment 37, wherein the brain permeability enhancer comprises cereport, regadenoson, or borneol.

39. The method of embodiment 38, wherein the brain permeability enhancer comprises cereport.

40. The method of embodiment 38, wherein the brain permeability enhancer comprises regadenoson.

41. The method of embodiment 38, wherein the brain permeability enhancer comprises borneol.

42. The method of any one of embodiments 1 to 41, which further comprises performing microbubble-enhanced ultrasound on the subject.

43. The method of any one of embodiments 1 to 42, wherein the BRD9 inhibitor is administered to the subject intranasally.

44. The method of any one of embodiments 1 to 42, wherein the BRD9 inhibitor is administered to the subject intravenously.

45. The method of any one of embodiments 1 to 42, wherein the BRD9 inhibitor is administered to the subject by intra-arterial injection.

46. The method of any one of embodiments 1 to 42, wherein the BRD9 inhibitor is administered to the subject's CSF.

47. The method of embodiment 46 wherein the BRD9 inhibitor is administered intrathecally, intracerebroventricularly or intraparenchymally.

48. A BRD9 inhibitor for use in the treatment of Huntington's Disease (HD) in a subject in need thereof.

49. The BRD9 inhibitor of embodiment 48, wherein the BRD9 inhibitor is not conjugated to a degron.

50. The BRD9 inhibitor of embodiment 48, wherein the administration of the BRD9 inhibitor does not induce proteasome-mediated degradation of BRD9 in vivo.

51. The BRD9 inhibitor of any one of embodiments 48 to 50, wherein the BRD9 inhibitor is a selective BRD9 inhibitor.

52. The BRD9 inhibitor of embodiment 51, wherein the BRD9 inhibitor has at least 2-fold or at least 5-fold greater inhibition of BRD9 as compared to BRD2.

53. The BRD9 inhibitor of embodiment 51 or embodiment 52, wherein the BRD9 inhibitor has at least 2-fold or at least 5-fold greater inhibition of BRD9 as compared to BRD3.

54. The BRD9 inhibitor of any one of embodiments 51 to 53, wherein the BRD9 inhibitor has at least 2-fold or at least 5-fold greater inhibition of BRD9 as compared to BRD4.

55. The BRD9 inhibitor of any one of embodiments 48 to 54, wherein the BRD9 inhibitor is a pyridinone compound.

56. The BRD9 inhibitor of any one of embodiments 48 to 54, wherein the BRD9 inhibitor is BI-9564:

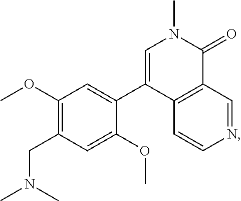 [SMILES: COc1cc(-c2cn(C)c(=O)c3cnccc23)c(OC)cc1CN(C)C]

or a pharmaceutically acceptable salt thereof.

57. The BRD9 inhibitor of any one of embodiments 48 to 54, wherein the BRD9 inhibitor is BI-7273:

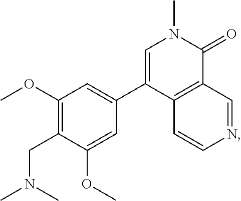 [SMILES: COc1cc(-c2cn(C)c(=O)c3cnccc23)cc(OC)c1CN(C)C]

or a pharmaceutically acceptable salt thereof.

58. The BRD9 inhibitor of any one of embodiments 48 to 54, wherein the BRD9 inhibitor is a methylquinolinone compound.

59. The BRD9 inhibitor of any one of embodiments 48 to 54, wherein the BRD9 inhibitor is LP-99:

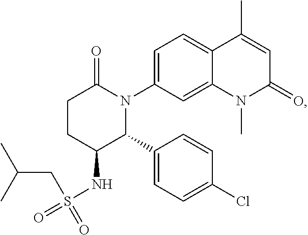 [SMILES: Cc1cc(=O)n(C)c2cc(N3C(=O)CC[C@H](NS(=O)(=O)CC(C)C)[C@H]3c3ccc(Cl)cc3)ccc12]

or a pharmaceutically acceptable salt thereof.

60. The BRD9 inhibitor of any one of embodiments 48 to 54, wherein the BRD9 inhibitor is a thienopyridone compound.

61. The BRD9 inhibitor of any one of embodiments 48 to 54, wherein the BRD9 inhibitor is I-BRD9:

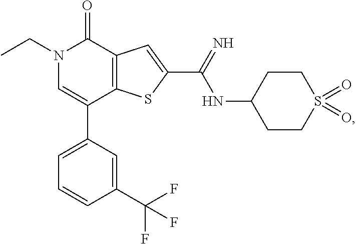 [SMILES: CCn1cc(-c2cccc(C(F)(F)F)c2)c2sc(C(=N)NC3CCS(=O)(=O)CC3)cc2c1=O]

or a pharmaceutically acceptable salt thereof.

62. The BRD9 inhibitor of any one of embodiments 48 to 54, wherein the BRD9 inhibitor is d-BRD9:

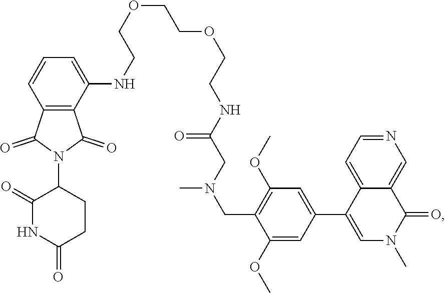 [SMILES: COc1cc(-c2cn(C)c(=O)c3cnccc23)cc(OC)c1CN(C)CC(=O)NCCOCCOCCNc1cccc2c1C(=O)N(C1CCC(=O)NC1=O)C2=O]

or a pharmaceutically acceptable salt thereof.

63. The BRD9 inhibitor of any one of embodiments 48 to 54, wherein the BRD9 inhibitor is TP-472:

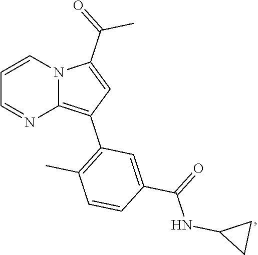 [SMILES: CC(=O)c1cc(-c2cc(C(=O)NC3CC3)ccc2C)c2ncccn12]

or a pharmaceutically acceptable salt thereof.

64. The BRD9 inhibitor of any one of embodiments 48 to 54, wherein the BRD9 inhibitor is GNE-375:

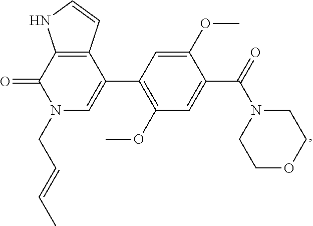 [SMILES: C/C=C/Cn1cc(-c2cc(OC)c(C(=O)N3CCOCC3)cc2OC)c2cc[nH]c2c1=O]

or a pharmaceutically acceptable salt thereof.

65. The BRD9 inhibitor of any one of embodiments 48 to 54, wherein the BRD9 inhibitor is a compound of Formula (I):

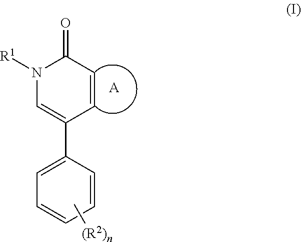

or an enantiomer, diastereomer, stereoisomer, or pharmaceutically acceptable salt thereof, wherein:
A is phenyl or 5- or 6-membered heteroaryl containing 1 or 2 heteroatoms selected from N and S, wherein the phenyl or heteroaryl is unsubstituted or substituted with 1 to 3 R³ groups;
R¹ is H, (C₁-C₄)alkyl, or (C₁-C₄)haloalkyl;
each R² is independently (C₁-C₄)alkyl, (C₁-C₄)haloalkyl, (C₁-C₄)alkoxy, (C₁-C₄)haloalkoxy, halogen, OH, or NH₂;
each R³ is independently (C₁-C₄)alkyl, (C₁-C₄)haloalkyl, (C₁-C₄)alkoxy, (C₁-C₄)haloalkoxy, halogen, OH, NH₂, or

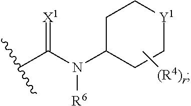

X₁ is NR⁵ or O;
Y₁ is S(O)ₐ or NR⁵;
each R⁴ is independently (C₁-C₄)alkyl, (C₁-C₄)haloalkyl, halogen, or —C(O)(C₁-C₃)alkyl;
each R⁵ is independently H or (C₁-C₄)alkyl;
each R⁶ is independently H or (C₁-C₄)alkyl;
a is 0, 1, or 2; and
n and r are each independently 0, 1, 2, or 3.

66. The BRD9 inhibitor of any one of embodiments 48 to 54, wherein the BRD9 inhibitor is a compound of Formula (II):

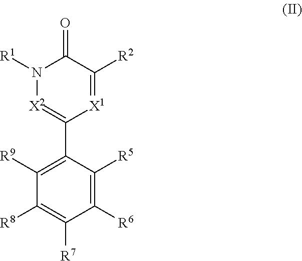

or an enantiomer, diastereomer, stereoisomer, or pharmaceutically acceptable salt thereof, wherein:
R¹ is (C₁-C₃)alkyl or cyclopropyl;
R² is halogen, (C₁-C₃)alkyl, (C₁-C₃)haloalkyl, NH₂, NH(C₁-C₃)alkyl or OH;
X¹ is N or CR³, and X² is N or CR⁴; provided that X¹ and X² cannot be both N;
R³ is H or (C₁-C₃)alkyl;
R⁴ is H or (C₁-C₃)alkyl; provided that R³ and R⁴ cannot be both (C₁-C₃)alkyl;
alternatively, R² and R³ taken together form a benzene ring or a 5-6 membered heteroarene ring, each of which rings can be independently unsubstituted or substituted with one or more groups which are independently halogen, OH, NH₂, NH(C₁-C₃)alkyl or (C₁-C₃)alkyl, wherein the (C₁-C₃)alkyl group can be unsubstituted or substituted with 5-6 membered heteroaryl or phenyl;
R⁵ and R⁹ are the same or different and are independently H, O(C₁-C₃)alkyl or (C₁-C₃)alkyl;
R⁶ and R⁸ are the same or different and are independently H, OH, halogen, NH₂, (C₁-C₃)alkyl, O(C₁-C₃)alkyl, O(C₁-C₃) haloalkyl, (C₁-C₃)alkyl-O—(C₁-C₃)alkyl, 4-7 membered heterocycloalkyl, (C₁-C₃)alkyl-SO₂—(C₁-C₃)alkyl, (C₁-C₃)alkyl-NH₂, (C₁-C₃)alkyl-N((C₁-C₃)alkyl)₂, N((C₁-C₃)alkyl)₂, or NHR¹³;
R¹³ is independently for each occurrence SO₂—(C₁-C₃)alkyl or (C₁-C₃)alkyl, wherein the (C₁-C₃)alkyl groups are unsubstituted or substituted with 5 to 6 membered heteroaryl;
alternatively, R⁵ and R⁶ taken together form a benzene ring;
alternatively, R⁷ and R⁶ or R⁷ and R⁸ taken together form a 5-7 membered heterocycloalkyl which is unsubstituted or substituted with (C₁-C₃)alkyl;
R⁷ is H, NH₂, Y—R¹², (C₁-C₃)alkyl or 4-7 membered heterocycloalkyl;
Y is CR¹⁰R¹¹, SO₂ or CO;
R¹⁰ and R¹¹ are the same or different and are independently H or (C₁-C₃)alkyl; or
R¹⁰ and R¹¹ taken together form a C₃₋₄ cycloalkyl;
R¹² is NH₂, OH, (C₁-C₃)alkyl, N(R¹⁵,R¹⁶), OR¹⁷, aryl, or 5-6 membered heteroaryl, wherein the aryl or heteroaryl are independently unsubstituted or substituted with one or more halogen or 4-7 membered heterocycloalkyl, each of which heterocycloalkyl is independently unsubstituted or substituted with one or more groups selected from halogen, OH, NH₂, (C₁-C₃)alkyl, NH(C₁-C₃)alkyl, N((C₁-C₃)alkyl)₂, O(C₁-C₃)alkyl and CH₂R¹⁴;
R¹⁴ is 5-10 membered mono- or bicyclic aryl or heteroaryl, which is unsubstituted or substituted with NH₂, OH, halogen, CN, (C₁-C₃)alkyl, or O(C₁-C₃)alkyl;
R¹⁵ is H or (C₁-C₃)alkyl;
R¹⁶ is (C₁-C₃)alkyl, C₂₋₃alkyl-N((C₁-C₃)alkyl)₂, C₂₋₃alkyl-NH(C₁-C₃)alkyl or 4-7 membered heterocycloalkyl, which heterocycloalkyl is unsubstituted or substituted with (C₁-C₃)alkyl;
R¹⁷ is (C₁-C₃)alkyl or 4-7 membered heterocycloalkyl, which heterocycloalkyl is unsubstituted or substituted with (C₁-C₃)alkyl;
wherein when R⁷ is YR¹², R⁶ and R⁸ can be the same or different and are independently H, OH, halogen, NH₂, CN, (C₁-C₃)alkyl, (C₁-C₃) haloalkyl, O(C₁-C₃)alkyl, O(C₁-C₃) haloalkyl or (C₁-C₃)alkyl-O—(C₁-C₃)alkyl; and
wherein at least one of the substituents R⁵ to R⁹ is not hydrogen.

67. The BRD9 inhibitor of any one of embodiments 48 to 54, wherein the BRD9 inhibitor is a non-selective BRD9 inhibitor.

68. The BRD9 inhibitor of embodiment 67, wherein the BRD9 inhibitor is bromosporine:

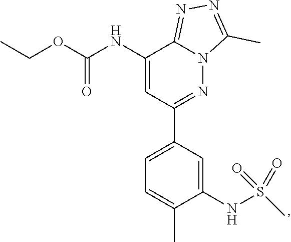 [SMILES: CCOC(=O)Nc1cc(-c2ccc(C)c(NS(C)(=O)=O)c2)nn2c(C)nnc12]

or a pharmaceutically acceptable salt thereof.

69. The BRD9 inhibitor of any one of embodiments 48 to 68, wherein the BRD9 inhibitor is administered in the form of an amino acid conjugate.

70. The BRD9 inhibitor of embodiment 69, wherein the amino acid conjugate is a lysine conjugate.

71. The BRD9 inhibitor of embodiment 69, wherein the amino acid conjugate is a phenylalanine conjugate.

72. The BRD9 inhibitor of any one of embodiments 48 to 71, wherein the BRD9 inhibitor is administered to the subject with a carrier.

73. The BRD9 inhibitor of embodiment 72, wherein the carrier comprises a nanoparticle, an exosome, or a carbon nanotube.

74. The BRD9 inhibitor of embodiment 73, wherein the carrier comprises a nanoparticle.

75. The BRD9 inhibitor of embodiment 74, wherein the nanoparticle comprises a lipid-based nanoparticle.

76. The BRD9 inhibitor of embodiment 74, wherein the nanoparticle comprises a human serum albumin-based nanoparticle.

77. The BRD9 inhibitor of embodiment 74, wherein the nanoparticle comprises an apolipoprotein-based nanoparticle.

78. The BRD9 inhibitor of embodiment 74, wherein the nanoparticle comprises a polymer-based nanoparticle.

79. The BRD9 inhibitor of embodiment 74, wherein the nanoparticle comprises a dendrimer-based nanoparticle.

80. The BRD9 inhibitor of embodiment 74, wherein the nanoparticle comprises an inorganic-based nanoparticle.

81. The BRD9 inhibitor of embodiment 73, wherein the carrier comprises an exosome.

82. The BRD9 inhibitor of embodiment 73, wherein the carrier comprises a carbon nanotube.

83. The BRD9 inhibitor of any one of embodiments 48 to 82, wherein treatment of the subject further comprises adjunctively administering a brain permeability enhancer to the subject.

84. The BRD9 inhibitor of embodiment 83, wherein the BRD9 inhibitor and the brain permeability enhancer are co-administered in a single pharmaceutical composition.

85. The BRD9 inhibitor of embodiment 83 or embodiment 84, wherein the brain permeability enhancer comprises cereport, regadenoson, or borneol.

86. The BRD9 inhibitor of embodiment 85, wherein the brain permeability enhancer comprises cereport.

87. The BRD9 inhibitor of embodiment 85, wherein the brain permeability enhancer comprises regadenoson.

88. The BRD9 inhibitor of embodiment 85, wherein the brain permeability enhancer comprises borneol.

89. The BRD9 inhibitor of any one of embodiments 48 to 88, wherein treatment of the subject further comprises performing microbubble-enhanced ultrasound on the subject.

90. The BRD9 inhibitor of any one of embodiments 48 to 89, wherein the BRD9 inhibitor is administered to the subject intranasally.

91. The BRD9 inhibitor of any one of embodiments 48 to 89, wherein the BRD9 inhibitor is administered to the subject intravenously.

92. The BRD9 inhibitor of any one of embodiments 48 to 89, wherein the BRD9 inhibitor is administered to the subject by intra-arterial injection.

93. The BRD9 inhibitor of any one of embodiments 48 to 89, wherein the BRD9 inhibitor is administered to the subject's CSF.

94. The BRD9 inhibitor of embodiment 93 wherein the BRD9 inhibitor is administered intrathecally, intracerebroventricularly or intraparenchymally.

95. Use of a BRD9 inhibitor in the manufacture of a medicament for the treatment of Huntington's Disease.

96. The use of embodiment 95, wherein the BRD9 inhibitor is a BRD9 inhibitor of any one of embodiments 49 to 94.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A method of treating a subject having Huntington's Disease (HD), comprising administering to the subject a therapeutically effective amount of a bromodomain 9 (BRD9) inhibitor.

2. The method of claim 1, wherein the wherein the BRD9 inhibitor is not conjugated to a degron.

3. The method of claim 1, wherein the administration of the BRD9 inhibitor does not induce proteasome-mediated degradation of BRD9 in vivo.

4. The method of claim 1, wherein the BRD9 inhibitor is a selective BRD9 inhibitor.

5-7. (canceled)

8. The method of claim 1, wherein the BRD9 inhibitor is a pyridinone compound.

9-10. (canceled)

11. The method of claim 1, wherein the BRD9 inhibitor is a methylquinolinone compound.

12. (canceled)

13. The method of claim 1, wherein the BRD9 inhibitor is a thienopyridone compound.

14-19. (canceled)

20. The method of claim 1, wherein the BRD9 inhibitor is a non-selective BRD9 inhibitor.

21-24. (canceled)

25. The method of claim 1, wherein the BRD9 inhibitor is administered to the subject with a carrier.

26-96. (canceled)
